(19) United States
(12) Patent Application Publication
(10) Pub. No.: US 2024/0345090 A1
(43) Pub. Date: Oct. 17, 2024

(54) EARLY DETECTION OF INNATE IMMUNE DYSFUNCTION AND TREATMENT OF CONDITIONS ASSOCIATED THEREWITH

(71) Applicant: UNIVERSITY OF FLORIDA RESEARCH FOUNDATION, INCORPORATED, Gainesville, FL (US)

(72) Inventors: Malú Mariadelourdes Gámez TANSEY, Gainesville, FL (US); Habibeh KHOSHBOUEI, Gainesville, FL (US)

(21) Appl. No.: 18/580,710

(22) PCT Filed: Jul. 19, 2022
(86) PCT No.: PCT/US22/37597

§ 371 Date: Jan. 19, 2024

Related U.S. Application Data
(60) Provisional application No. 63/223,353, filed on Jul. 19, 2021.

(51) Int. Cl. G01N 33/573; A61K 38/00; C07K 14/525; G01N 33/68

(52) U.S. Cl. CPC G01N 33/573; C07K 14/525; G01N 33/6854; A61K 38/00; G01N 2333/90245; G01N 2800/50

(57) ABSTRACT

Disclosed is a method for detecting a level of tyrosine hydroxy lase (TH) in a biosample from a subject. The biosample comprises a homogenate of peripheral monocytes from the subject. Detection involves conducting an ELISA on the biosample using a biotinylated anti-TH antibody under conditions to allow the biotinylated anti-TH antibody to bind to TH. If elevated TH is detected, an amount of a TNFα inhibitor effective to decrease TH in peripheral monocytes of the subject may be administered. In a particular method, a TNFα inhibitor is administered if the TH level in the biosample is higher than a threshold level.

### BACKGROUND

Human and animal studies have shown that most if not all immune cells possess components necessary to release, uptake, synthesize, and respond to catecholamines including dopamine and norepinephrine (NOR). These components activate signaling cascades that change the phenotype and function of cells in both healthy and in disease conditions. Immune cells may thus both come in contact with physiological levels of catecholamines derived from peripheral tissues and also serve as a source for catecholamines. Tyrosine hydroxylase (TH) catalyzes the conversion of tyrosine to 3,4-dihydroxyphenylalanine (L-DOPA), which is the rate-limiting step in the synthesis of dopamine, norepinephrine (NOR) and epinephrine^{1,2}. Although primarily studied in the central nervous system^{3,4}, TH is expressed in the majority of peripheral immune cells⁵⁻⁹, and many peripheral tissues¹⁰, including kidney^{11,12}, heart¹³ and adrenal cortex¹⁴⁻¹⁶. Both myeloid and lymphoid lineages of human peripheral immune cells express TH^{17,18}, which is thought to regulate dopamine levels within these cells⁹. Beyond protein expression, TH activity is regulated by a variety of post-translational modifications and that can regulate TH function. For example, phosphorylation, ubiquitination, nitration and S-glutathionlyation can all affect TH activity independent of TH levels¹⁹⁻²⁶. As the key to catecholamine production, TH activity and its relative expression are commonly studied in diseases in which catecholamine tone, synthesis and signaling are altered. These disease states include bipolar disorder, addiction, schizophrenia, attention deficit hyperactivity (ADHD) and neurodegenerative conditions including Parkinson's disease (PD).

The lack of a robust and sensitive assay to measure low levels of TH protein has hampered the field's ability to investigate TH protein levels in peripheral immune cells in diseases characterized by altered catecholamine tone. For example, in PD, due to its spatially restricted expression, decreases in TH levels in the basal ganglia are readily detectable^{27,28}, whereas changes in TH levels in other brain regions (i.e., amygdala, hippocampus, cortical regions) are reported in the later stages of PD^{29,30}. In contrast, very low TH levels in countless immune cells spread across the body has made it difficult to study TH protein levels in peripheral immune cells. For example, indirect TH measurements via qPCR reveal that PD patients show significantly less midbrain TH mRNA compared to healthy controls subjects (5.5±1.4 in healthy controls, vs. 1.5±0.9 attomole/microgram total RNA in PD)³¹. In contrast, TH mRNA is not detectable in unstimulated immune cells³². TH protein expression in the substantia nigra is in excess of 200 ng TH per milligram protein³³, and is decreased in patients with PD. However, to our knowledge no reports directly quantify TH protein in immune cells.

### BRIEF DESCRIPTION OF THE DRAWINGS

FIG. 1. Establishing a reproducible quantitative Bio-ELISA to detect Tyrosine Hydroxylase. A-D) FIG. 1A shows a diagram of the general ELISA strategy. TH is detectable in recombinant form and in PC12 crude lysate using affinity purified rabbit polyclonal TH antibody AB152 (Sigma) (FIG. 1B), and antibodies selected for this ELISA, mouse monoclonal MCA-4H2 (EnCor) (FIG. 1C) and rabbit polyclonal RPCA-TH (EnCor) (FIG. 1D). Using AB152, the lower threshold for TH detection was probed via serial dilution of purified recombinant TH from 6 ug/mL to 0.094 ug/mL followed by Western blot and near-Infrared detection, considered to be a sensitive method for protein detection on Western blot (FIG. 1E). It was demonstrated that IR detection is reliable to a lower threshold of ˜15 ng TH. Below this limit, TH detection becomes unreliable with IR detection. In a series of stepwise experiments (steps shown in FIG. 1F, FIG. 1G, and FIG. 1H) designed to increase ELISA sensitivity and decrease background, lower detection limits of 15 pg/mL TH were achieved. Capture antibody and detection antibody in all three methods were MCA-4H2 (1:1,000 dilution from 1 mg/mL) and RPCA-TH (1:6,000 dilution from 1 mg/mL). Schematic representation of each method shown on the left with representative standard curve on the right. Incubation with detection antibody followed by an HRP-conjugate secondary yielded a lower detection threshold of 125 pg/mL (FIG. 1F). Addition of a tertiary layer using anti-rabbit biotin followed by Avidin-HRP improved lower detection threshold to 62.5 pg/mL but resulted in increased background (FIG. 1G). Use of biotinylated detection antibody (RPCA-TH-biotin, 1:6,000 dilution from 1.65 mg/mL) followed by avidin-HRP yielded the lowest detection threshold of 15 pg/mL, with maximum sensitivity and minimal background (FIG. 1H). For FIGS. 1F, 1G and 1H, insets (red outline) shows magnified lower standard curve to illustrate sensitivity.

FIG. 2. Antibodies MCA-4H2 and RPCA-TH reliably detect both native and denatured TH in mouse and human tissue. Human and murine brain sections (40 um) were permeabilized, blocked and stained with primary antibodies (MCA-4H2 and RPCA-TH) followed by HRP-conjugated secondaries and detected using diaminobenzidine enhanced with nickel (NiDAB, grey-black). FIG. 2A: MCA-4H2 stains neuromelanin-expressing (brown) TH positive midbrain neurons and neuronal processes (grey-black) with no non-specific staining (secondary only, top panel; isotype control, second panel) in both human and murine tissues. FIG. 2B: RPCA-TH shows similar highly specific staining of midbrain TH positive neurons, confirming antibody specificity. A & B) Human midbrain tissues shown as secondary-only and isotype controls exhibit endogenous neuromelanin (brown), not to be confused with immunostaining. FIG. 2C: Western blot analyses of murine and human striatal tissues reveal similarly specific detection of TH (˜63 kDa band) in both mouse and human, with minimal non-specific staining in negative control homogenate (parental CHO cell homogenate). (Left-MCA-4H2, right-RPCA-TH). FIG. 2D: Blocking peptide/absorption control followed by western blot detection with either RPCA-TH and MCA-4H2 confirms specificity of both antibodies for TH protein. HSP60 (loading control) is shown below and applies to C-D.

FIG. 3. Bio-ELISA reliably quantifies TH in PC12 cells, human macrophages and cultured murine dopamine neurons. FIG. 3A: Using the Bio-ELISA shown in FIG. 1G, TH was quantified in four relevant tissues and cultured cells: PC12 (positive control), HEK293 (negative control), cultured human macrophages and cultured primary murine dopamine neurons. PC12 cells express very high levels of TH (<10 ng TH/mg total protein) relative to human macrophages (˜300 pg TH/mg total protein) and primary murine dopamine neurons (˜700 pg TH/mg total protein). FIG. 3B: TH values are plotted on a representative standard curve for visual comparison, with inset magnifying the lower end of the standard curve. FIG. 3C: Calculations are shown by which raw TH concentration in ng/ml is normalized to total protein per sample. Samples included in A, each an independent biological replicate, are shown in C. Data are shown as ±SEM.

FIG. 4. Absorption controls demonstrate specificity of TH Bio-ELISA. FIG. 4A: Schematic layout of experimental conditions to assess absorption controls in contrast to optimized Bio-ELISA conditions using PC12 cell lysate. FIG. 4B: Representative standard curve shown to illustrate PC12 cells' TH concentration using optimized Bio-ELISA (blue arrow), absorbed capture antibody (MCA-4H2 preincubated with 20 ug/mL recombinant TH, orange arrow), and absorbed detection antibody (biotinylated RPCA-TH preincubated with 20 ug/mL recombinant TH, green arrow). PC12 TH is undetectable after absorption of either capture or detection antibodies, confirming assay specificity.

FIG. 5. TH protein is increased in CD14+ monocytes isolated from PD patients. Total CD14+ monocytes were magnetically isolated from 20 million freshly isolated PBMCs derived from whole blood of 11 PD patients and 11 healthy volunteers, immediately lysed in the presence of protease inhibitor and stored at −80° C. Following protein quantification, whole lysate from each sample was added to duplicate wells and assayed for concentration of TH. FIG. 5A; Monocytes isolated from PD patients express significantly greater quantity of TH compared to equivalent monocytes isolated from healthy control subjects (unpaired two-tailed T-test, alpha=0.05, p<0.05). FIG. 5B: Mean TH concentration for monocytes from PD patients plotted on a representative standard curve, with inset magnifying the lower end of the curve. FIG. 5C: Calculations are shown by which raw TH concentration in ng/ml is normalized to total protein per sample. Samples included in A, each an independent biological replicate, are shown in C. Data are shown as ±SEM.

FIG. 6. TNFα increases number of TH+ monocytes and amount of TH protein per monocyte. FIG. 6A: Total CD14+ monocytes were isolated using negative magnetic selection from 80 million healthy donor PBMCs. Monocytes were seeded into a 6-well ultra-low-adherence plate at 2 million cells per well, and treated with vehicle (media), TPA (100 ng/ml, positive control), TNFα (17 ng/ml), in duplicate. FIG. 6B: One duplicate was assayed by flow cytometry to detect TH-expressing monocytes, using counting beads as a reference value to quantify the number of TH+ cells. FIG. 6C: Number of TH+ cells were quantified as shown. FIG. 6D: Representative histogram showing one set of samples assayed for TH expressing monocytes following stimulation. FIG. 6E: Both TPA and TNFα induced significant increases in TH-expressing monocytes relative to vehicle, shown as fold-increase relative to vehicle (n=3 per group, one-way ANOVA, p<0.01). FIG. 6F: No increase in total monocytes per condition, relative to vehicle (n=3 per group, one-way ANOVA, n.s.). FIG. 6G: TH concentration in picograms per milligram total protein shows TNFA treatment results in significantly increased TH protein relative to vehicle and TPA (n=5-6 per group, one-way ANOVA, p<0.001). FIG. 6H: Mean TH protein level for monocytes treated with vehicle, TPA and TNFα are plotted on a representative standard curve, with the inset magnifying the lower end of the curve. FIG. 6I: Intracellular flow cytometry for Ki67 does not reveal significant differences between vehicle and TNFα treatment groups, confirming a lack of cell proliferation following TNFα treatment. Data are shown as ±SEM.

FIG. 7. Inhibition of TNFα blocks increase in number of TH+ monocytes and amount of TH per monocyte. Acutely isolated monocytes from three healthy donors (FIG. 7A) were seeded at 2 million cells per well in duplicate ultra-low-adherence plates, and treated with TNFα (17 ng/ml), XPro1595 (50 ng/ml), IL6 (17 ng/ml) or combinations thereof as indicated (FIG. 7B). FIG. 7C: In samples assayed by flow cytometry, using counting beads as a reference value to quantify the number of TH+ cells, co-incubation with TNFα and XPro1595 significantly reduced the number of TH+ monocytes relative to TNFα treatment alone. Treatment with IL6 or IL6+XPro1595 resulted in no significant change in the number of TH+ monocytes. Values are represented as fold change relative to vehicle (n=3 per group, one way ANOVA, *P<0.05, **P<0.01). FIG. 7D: TH concentration (picogram per milligram total protein) significantly increases upon TNFα treatment, and is reduced significantly to baseline levels following co-incubation with TNFα and XPro1595. Neither IL6 nor IL6+XPro1595 significantly increased TH quantity (n=3 per group, one way ANOVA, *P<0.05). Data are shown as ±SEM.

### GENERAL DESCRIPTION

As is disclosed herein, levels of tyrosine hydroxylase (TH) in peripheral monocytes of patients who have a neurological disorder such as Parkinson's disease are elevated versus samples from healthy subjects. It is also disclosed herein that the level of TH in peripheral monocytes is mediated by TNFα soluble form and that inhibiting TNFα soluble form reduces TH in peripheral monocytes. Provided is an exceptionally sensitive assay to detect very low levels of TH in peripheral monocytes of a subject. Levels of TH in peripheral monocytes of neurological disorder subjects are consistently higher than control samples. Accordingly, embodiments provided herein involve the detection of subjects who are at high risk of developing a neurological disorder or other disorder associated with innate immune dysfunction (e.g. atherosclerosis and metabolic syndrome) and a mode of treating those subjects to delay the onset or prevent onset of such disorders.

### Overview

In order to investigate whether the characteristically reduced TH expression in PD central nervous system (CNS) is recapitulated in peripheral immune cells, we established a sensitive assay to quantify TH protein. We then applied the assay to analyze TH production in peripheral blood monocytes. The sensitivity of our Bio-ELISA was a thousand-fold above traditional detection methods, and when we measured TH level in peripheral monocytes from healthy controls and from PD, we observed a significant elevation of TH levels in PD monocytes versus controls. This observation was contrary to our a priori hypothesis. The unexpected discovery of increased TH protein in peripheral PD monocytes prompted investigation into the potential underlying mechanism. In the PD literature, there is a strong consensus that neuroinflammatory cytokines, including TNFα and IL6 are increased in CSF and serum of PD patients and of animal models of PD^{27,34-42}. Therefore, we investigated whether ex vivo exposure to TNFα or IL6 increases the number of TH+ monocytes and/or amount of TH protein per monocyte. We found that exposure to TNFα, but not IL6 increased both the number of TH+ monocytes and the quantity of TH protein per cell.

### Definitions

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and intended to be non-limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention are performed generally according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwartz, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

The terms “administering” or “administration” of an agent, drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, intradermally or subcutaneously), rectally, or topically. Administering or administration includes self-administration and the administration by another.

The term “biosample” as used herein refers to a sample obtained from a subject. A biosample may include any fluid or tissue sample that comprises PBMCs, or lysates of any of the foregoing.

The term “innate immune dysfunction” refers to a state of the innate immune system associated with elevated baseline inflammation such as a basal elevation in pro-inflammatory mediators. Innate immune dysfunction also is often paradoxically associated with a weakened response to immune challenge thereby making subjects susceptible to infection. It is known that innate immune dysfunction increases with age. Brubaker et al., Aging Dis, 2011 Oct. 2 (5): 346-360. Innate immune dysfunction can lead to other disorders or diseases such as autoimmune disorders, cancer, metabolic syndrome, artherosclerosis, arthritis and neurodegenerative disorders (e.g. Alzheimers disease and Parkinson's disease) as well as chronic inflammation. The present disclosure contemplates identifying individuals who may already be exhibiting markers of innate immune dysfunction before other associated disorders manifest.

The term “peripheral blood mononuclear cells (PBMCs) refer to blood cells with round nuclei, such as monocytes, lymphocytes, and macrophages. In a specific example PBMCs refer to monocytes.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The terms “pharmaceutically acceptable carrier, excipient, vehicle, or diluent” refer to a medium which does not interfere with the effectiveness or activity of an active ingredient and which is not toxic to the hosts to which it is administered. A carrier, excipient, vehicle, or diluent includes but is not limited to binders, adhesives, lubricants, disintegrates, bulking agents, buffers, and miscellaneous materials such as absorbents that may be needed in order to prepare a particular composition.

The term “subject” as used herein refers to an individual. For example, the subject is a mammal, such as a primate, and, more specifically, a human. The term does not denote a particular age or sex. Thus, adult and newborn subjects, whether male or female, are intended to be covered. In a specific embodiment, the subject include a subject in need.

The term “subject in need” refers to a subject that exhibits symptoms, characteristics or markers of innate immune dysfunction. In a specific embodiment, the subject in need is one that exhibits elevated TH (i.e. above a threshold level) in peripheral monocytes obtained from the subject.

As used herein, “therapeutically effective amount” or “an effective amount” have the standard meanings known in the art and are used interchangeably herein to mean an amount sufficient to treat a subject afflicted with a disease (e.g., innate immune dysfunction) or to alleviate a symptom or a complication associated with the disease, or to decrease TH levels in peripheral monocytes of a subject.

### DESCRIPTION OF CERTAIN EMBODIMENTS

According to one embodiment, disclosed is a method for detecting a level of tyrosine hydroxylase (TH) in a biosample from a subject. The biosample comprises a homogenate of peripheral monocytes from the subject. Detection involves conducting an ELISA on the biosample using a biotinylated anti-TH antibody under conditions to allow the biotinylated anti-TH antibody to bind to TH. If elevated TH is detected, an amount of a TNFα inhibitor effective to decrease TH in peripheral monocytes of the subject may be administered. In a particular method, a TNFα inhibitor is administered if the TH level in the biosample is higher than a threshold level. A threshold level may comprises a level that is at least 10% higher than a biosample of peripheral monocytes from a healthy subject. Also, a threshold level may be a defined level such at least 15 pg TH, at least 20 pg TH, at least 25 pg TH, at least 30 pg TH, at least 35 pg TH, at least 40 pg TH, at least 45 pg TH, at least 50 pg TH, at least 55 pg/TH, at least 60 pg/TH at least 70 pg TH, at least 80 pg TH, at least 90 pg TH, at least 100 pg/TH at least 125 pg/TH or at least 150 pg TH per mg protein in the biosample.

The amount of the TNFα inhibitor administered is an amount effective to reduce TH in peripheral monocytes by at least 10, at least 20, at least 30, at least 40, at least 50, at least 60, at least 70, at least 80 or at least 90 percent. An additional detection step may be conducted following administration of the TNFα inhibitor.

According to another embodiment, a method is provided for treating innate immune dysfunction in a subject that involves administering an amount of a TNFα inhibitor effective to decrease TH in peripheral monocytes of the subject if peripheral monocytes of the subject comprise a threshold level of TH.

According to a specific embodiment, provided is a method comprising: obtaining a homogenate peripheral monocytes from a subject; subjecting the homogenate to a biotinylated anti-Tyrosine hydroxylase (TH) antibody to form a homogenate antibody combination; subjecting the combination to horse radish peroxidase conjugated with avidin (HRP-avidin), wherein contact between HRP-avidin and biotinylated anti-TH antibody is made under conditions to produce a colorimetric signal; measuring the colorimetric signal, wherein a signal meets a threshold level indicates innate immune dysfunction in the subject. In one specific example, the threshold level may comprise a level that is at least 10% higher than that in a homogenate of peripheral monocytes from a healthy subject. In another example, the threshold level is at least 15 pg TH, at least 20 pg TH, at least 25 pg TH, at least 30 pg TH, at least 35 pg TH, at least 40 pg TH, at least 45 pg TH, at least 50 pg TH, at least 55 pg/TH, at least 60 pg/TH at least 70 pg TH, at least 80 pg TH, at least 90 pg TH, at least 100 pg/TH at least 125 pg/TH or at least 150 pg TH per mg protein in the homogenate.

### TNFα Soluble Form Inhibitors

The term TNFα soluble form inhibitor as used herein relates to an inhibitor that decreases the level or activity of TNFα soluble form. Examples of TNFα soluble form inhibitors include dominant-negative inhibitors such as described in Zalevsky et al, Journal of Immunology, Aug. 1, 2007, 179 (3) 1872-1883; and U.S. Pat. Pub 20040170602 (both incorporated herein in their entirety), and human recombinant mAbs such as adlimumab, infliximab, golimumab, or certolizumab, or dimeric fusion proteins of a portion of the extracellular ligand-binding portion of TNF receptor linked to the Fc portion of an IgG1 (e.g. etanercept). In a specific example, the TNFα soluble form inhibitor is XPRO 1595. XPRO-1595 is described in Table 1 of PCT Pub. No. WO2022/115179, which is incorporated herein by reference.

In further specific examples, the TNFα inhibitor relates to human SOITNF (UniProtKB/Swiss-Prot database entry P01375) with amino acid substitutions Y87H/A145R or I97T/A145R. Such TNF-alpha inhibitors may also include further modifications such as having (1) N-terminal MHHHHHH, amino acid substitution R31C, postranslational modification (Mod) of amino acid C31, and/or pegylation, e.g. conjugation of mPEG, monomethoxy-polyethylene glycol. The human solTNF is provided below as SEQ ID NO: 1:
VRSSSRTPSDKPVAHVVANPQAEGQLQWLNRRANALLANGVELRDNQLVVPS EGLYLIYSQVLFKGQGCPSTHVLLTHTISRIAVSYQTKVNLLSAIKSPCQRETPEGAEAK PWYEPIYLGGVFQLEKGDRLSAEINRPDYLDFAESGQVYFGIIAL

Other human solTNF inhibitors pertain to variants of SEQ ID NO: 1. It should be noted, that unless otherwise stated, all positional numbering of variant solTNF proteins is based on SEQ ID NO:1. That is, as will be appreciated by those in the art, an alignment of solTNF proteins and variant solTNF proteins may be done using standard programs, as is outlined below, with the identification of “equivalent” positions between the two proteins. Thus, the variant solTNF proteins are non-naturally occurring; that is, they do not exist in nature.

In some embodiments, the variant solTNF protein comprises non-conservative modifications (e.g. substitutions) to SEQ ID NO: 1. By “nonconservative” modification herein is meant a modification in which the wild type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. In a preferred embodiment, the variant TNFSF proteins of the present invention have at least one nonconservative modification.

Conservative modifications are generally those shown below, however, as is known in the art, other substitutions may be considered conservative: 1

### Ala Ser

Arg Lys
Asn Gln, His
Asp Glu
Cys Ser
Gin Asn
Glu Asp
Gly Pro
His Asn, Gin
Ile Leu, Val
Leu Ile, Val
Lys Arg, Gin, Glu
Met Leu, Ile
Phe Met, Leu, Tyr
Ser Thr
Thr Ser
Trp Tyr
Tyr Trp, Phe
Val Ile, Leu

Accordingly, in certain examples, the TNFα inhibitor pertains to a variant solTNF that comprises one or more conservative substitutions, optionally in addition to substitutions Y87H/A145R or I97T/A145R.

The variant proteins may be generated, for example, by using a PDA™ system previously described in U.S. Pat. Nos. 6,188,965; 6,296,312; 6,403,312; U.S. Ser. Nos. 09/419,351, 09/782,004, 09/927,790, 09/877,695, and 09/877,695; alanine scanning (see U.S. Pat. No. 5,506,107), gene shuffling ((WO 01/25277), site saturation mutagenesis, mean field, sequence homology, or other methods known to those skill in the art that guide the selection of point mutation sites and types.

In a preferred embodiment, sequence and/or structural alignments may be used to generate the variant TNFSF proteins for use in embodiments described herein. As is known in the art, there are a number of sequence-based alignment programs; including for example, Smith-Waterman searches, Needleman-Wunsch, Double Affine Smith-Waterman, frame search, Gribskov/GCG profile search, Gribskov/GCG profile scan, profile frame search, Bucher generalized profiles, Hidden Markov models, Hframe, Double Frame, Blast, Psi-Blast, Clustal, and GeneWise. There are also a wide variety of structural alignment programs known. See for example VAST from the NCBI (ncbi.nlm.nih.gov: 80/Structure/VAST/vast.shtml); SSAP (Orengo and Taylor, Methods Enzymol 266 (617-635 (1996)) SARF2 (Alexandrov, Protein Eng 9 (9): 727-732. (1996)) CE (Shindyalov and Bourne, Protein Eng 11 (9): 739-747. (1998)); (Orengo et al., Structure 5 (8): 1093-108 (1997); Dali (Holm et al., Nucleic Acid Res. 26 (1): 316-9 (1998), all of which are incorporated by reference).

### Compositions, Administration, Routes, and Doses of Vaccines

TNFα inhibitors disclosed herein are contemplated for administration to a subject in need, and can be administered by any convenient method known to the person of skill in the art. Administration can be by any route, including but not limited to local and systemic methods, for example aerosols for delivery to the lung, oral, rectal, vaginal, buccal, transmucosal, intranodal, transdermal, subcutaneous, intravenous, subcutaneous, intradermal, intratracheal, intramuscular, intraarterial, intraperitoneal, intracranial (e.g., intrathecal or intraventricular) or any known and convenient route. Preferred routes of administration are intravenous, intraperitoneal, subcutaneous, and/or oral/nasal administration. The form of the administration can determine how the TNFα inhibitor is formulated, and this is easily determined by the skilled artisan.

Compositions embodiments comprising TNFα inhibitors therefore can include, but are not limited to, solid preparations for oral administration, solid preparations to be dissolved in a liquid carrier for oral or parenteral administration, solutions, suspensions, emulsions, oils, creams, ointments, lotions, gels, powders, granules, cells in suspension, and liposome-containing formulations, and the like, or any convenient form known in the art. These compositions may be generated from a variety of components that include, but are not limited to, preformed liquids, self-emulsifying solids and self-emulsifying semisolids.

Solutions or suspensions used for parenteral, intradermal, subcutaneous or other injection can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

TNFα inhibitor containing compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that they can pass through a syringe and needle easily enough for administration. It should be stable under the conditions of manufacture and storage and should be preserved against the contaminating action of microorganisms such as bacteria and fungi. All solutions used to solubilize DNA or RNA should also be DNase-free and RNase-free.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions comprising one or more disclosed TNFα inhibitors can be prepared by incorporating the active agent in the required amount in an appropriate solvent with one or a combination of the ingredients enumerated above, as required, followed by filter sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. The skilled person is aware of how to use these dried preparations for injection.

Oral compositions comprising one or more disclosed TNFα inhibitors generally include an inert diluent or an edible carrier. They can be enclosed in gelatin capsules or compressed into tablets. Depending on the specific conditions being treated, pharmaceutical compositions of the present invention for treatment of innate immune dysfunction can be formulated and administered systemically or locally. Techniques for formulation and administration can be found in “Remington: The Science and Practice of Pharmacy” (20th edition, Gennaro (ed.) and Gennaro, Lippincott, Williams & Wilkins, 2000). For oral administration, the TNFα inhibitor can be contained in enteric forms to survive the stomach or further coated or mixed to be released in a particular region of the GI tract by known methods. For the purpose of oral therapeutic administration, the TNFα inhibitor can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL® or corn starch; a lubricant such as magnesium stearate or STEROTES®; a glidant such as colloidal silicon dioxide; a sweetening agent such as sucrose or saccharin; or a flavoring agent such as peppermint, methyl salicylate, or orange flavoring.

Systemic administration can also be by transmucosal means to the intestinal or colon, such as by suppository or enema, for example. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the disclosed nucleic acid constructs are formulated into ointments, salves, gels, or creams as generally known in the art.

In several embodiments, the disclosed TNFα inhibitors are prepared with carriers that will protect the compound against rapid elimination from the body, such as a controlled release or delayed formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art.

Formulations comprising one or more disclosed TNFα inhibitors designed to provide extended or delayed release also are contemplated for use with the invention. The following United States patents contain representative teachings concerning the preparation of uptake, distribution and/or absorption assisting formulations: U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756. Such compositions are contemplated for use with the invention.

The pharmaceutical formulations comprising one or more disclosed TNFα inhibitors, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. The active agents described herein also can be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Such methods for creating liquid, solid, semi-solid, gel, powder or inhalable formulations and the like are known in the art. Techniques for formulation and administration can be found in “Remington: The Science and Practice of Pharmacy” (20th edition, Gennaro (ed.) and Gennaro, Lippincott, Williams & Wilkins, 2000). Alternatively, the inventive compounds can be fused to microspheres in suspension for intravenous injection.

Dosages and regimens for administration are determined by the person of skill, including physicians. Administration of compositions, including the TNFα inhibitor composition can be performed a single time, or repeated at intervals, such as by continuous infusion over a period of time, four times daily, twice daily, daily, every other day, weekly, monthly, or any interval to be determined by the skilled artisan based on the subject involved. Treatment can involve administration over a period of one day only, a week, a month, several months, years, or over a lifetime. Regimens and duration can vary according to any system known in the art, as is known to the skilled person.

Doses of the disclosed TNFα inhibitors can be determined by the skilled artisan based on the condition of the subject and the route of administration to be used, but are expected to range from about 100 μg to about 10 mg, preferably from about 500 μg to about 10 mg, or about 1 mg to about 10 mg, or about 1 mg to about 5 mg or about 5 mg to about 10 mg and most preferably from about 1 mg to about 5 mg. Optimization/pharmacokinetics can make lower doses effective, therefore even lower doses are contemplated for use with the invention, for example about 10 μg to about 100 μg.

### EXAMPLES

### Example 1: Methods

Human subjects: Human brain tissues were obtained via approved IRB protocols #IRB201800374 and IRB202002059 respectively. Blood samples were obtained at the University of Florida Center for Movements Disorders and Neurorestoration according to an IRB-approved protocol (#IRB201701195).

Brain Tissues from Healthy Subjects

Human brain tissues were obtained via approved IRB protocols IRB202002059 and IRB201800374, from the UF Neuromedicine Human Brain and Tissue Bank (UF HBTB). The tissues were not associated with identifying information, exempt from consent, therefore no consent was required. Regions of interest were identified and isolated by a board-certified neuropathologist.

Blood Samples from Healthy Subjects

Blood samples from age-matched healthy subjects were obtained from two sources: an approved IRB protocol with written informed consent (IRB201701195), or were purchased from Lifesouth Community Blood Center, Gainesville, FL from August 2017 to January 2020 as deidentified samples, and exempt from informed consent (IRB201700339). According to Lifesouth regulations, healthy donors were individuals aged 50-80 years-old of any gender, who were not known to have any blood borne pathogens (both self-reported and independently verified), and were never diagnosed with a blood disease, such as leukemia or bleeding disorders. In addition, none of the donors were using blood thinners or antibiotics, or were exhibiting signs/symptoms of infectious disease, or had a positive test for viral infection in the previous 21 days.

Blood Samples from PD Patients:

Blood samples were obtained from PD patients (aged 50-80 years-old of any gender) at the University of Florida Center for Movements Disorders and Neurorestoration according to an IRB-approved protocol (#IRB201701195), via written informed consent. All recruited patients' PD was idiopathic. Patients did not have any recorded blood-borne pathogens or blood diseases, nor were they currently taking medications for infections according to their medical record. In addition, none of the donors were using blood thinners (warfarin, heparin), antibiotics, over-the-counter (OTC) medications other than aspirin, or were exhibiting signs/symptoms of infectious disease or had a positive test for viral infection in the previous 21 days.

### TH Recombinant Protein

Full length human TH protein was expressed from a synthetic cDNA inserted into the EcoRI and SalI sites of the pET30a (+) vector and was codon optimized for expression in E. coli. The vector adds an N-terminal His-tag and other vector sequence, a total of 5.7 kDa. Expression of the construct was made by standard methods and purification was performed using the His tag by immobilized metal affinity chromatography on a nickel column. The TH sequence used in this study is the human tyrosine 3-monooxygenase isoform shown in Uniprot entry P07101-2.

### Model Systems Used for the Validation of Bio-ELISA

Human macrophages: Primary human macrophages were cultured as described previously⁴³. Peripheral blood mononuclear cells (PBMCs) isolated as described below were re-suspended in RPMI 1640 containing 1% Pen/Strep and 7.5% sterile-filtered, heat-inactivated autologous serum isolated from the donor's own blood, and plated in 24-well untreated polystyrene plates at 1 million PBMCs per well. To retain only monocytes/macrophages, cells were washed after 90 minutes of adherence time to remove non-adherent cells with incomplete RPMI 1640, followed by replacement with complete media. Media was replaced at days 3 and 6 following culture, and cell lysis performed on day 7 following culture.

Primary murine midbrain dopamine neurons: Midbrain dopamine neurons strongly express TH⁴⁴ and were used as a positive control group. Animal studies were performed in compliance with University of Florida IACUC ethical regulations and rules (IACUC #201808953). Acutely dissociated mouse midbrains from 0-2 day-old male and female pups were isolated and incubated in dissociation medium at 37° C. under continuous oxygenation for 90 minutes. Dissociated cells were pelleted by centrifugation at 1,500×g for 5 min and resuspended and triturated in glial medium (Table 1). Cells were then plated on 12 mm coverslips coated with 0.1 mg/ml poly-D-lysine and 5 μg/ml laminin and maintained in neuronal media. Every 4 days, half the media was replaced with fresh media. The materials used for the preparation and maintenance of midbrain neuronal culture are outlined in Table 1.

TABLE 1
Neuron culture reagents
Dissociation Media

| Chemical name | Concentration | | Vendor | Catalog Number |
|---|---|---|---|---|
| NaCl | 116 | mM | Sigma- Aldrich | S7653 |
| NaHCo₃ | 26 | mM | Sigma- Aldrich | D6546 |
| NaH₂PO₄ | 2 | mM | Sigma- Aldrich | S9638 |
| D-glucose | 25 | mM | Sigma- Aldrich | G8769 |
| MgSO₄ | 1 | mM | Sigma- Aldrich | M7506 |
| Cysteine | 1.3 | mM | Sigma- Aldrich | C7352 |
| Papain | 400 | units/ml | Worthington | LS003127 |
| Kynurenic acid | 0.5 | mM | Sigma- Aldrich | K3375 |

Glia Media

| | | | |
|---|---|---|---|
| DMEM | 51.45 | Thermo Fisher Scientific | 11330032 |
| Fetal Bovine Serum | 39.60 | Gemini | 100-106 |
| Penicillin/Streptomycin | 0.97 | Thermo Fisher Scientific | 15-140-122 |
| Glutamax 100X | 0.97 | Thermo Fisher Scientific | 35050061 |
| Insulin (25 mg/ml stock) | 0.08 | Sigma-Aldrich | I5500 |

Neuronal Media

| | | | |
|---|---|---|---|
| Neurobasal-A | 96.9 | Thermo Fisher Scientific | 10888022 |
| B27 Plus | 1.9 | Thermo Fisher Scientific | A3582801 |
| GDNF | 0.97 | Sigma-Aldrich | SRP3200 |
| Glutamax 100X | 0.15 | Thermo Fisher Scientific | 35050061 |
| Kynurenic acid | 0.08 | Sigma-Aldrich | K3375 |

Positive and negative control cell lines: All cell cultures were maintained at 37° C. with 5% CO₂ and all cell culture supplies are listed in Table 2. HEK293 cells⁴⁵ are not thought to express TH and so were used as a negative expression control and were cultured as described previously^{46,47}. PC12 cells express TH⁴⁸ and were used as a positive control. The cells were cultured as described by Cartier et al. 2010⁴⁹. CHO cells were cultured as previously described⁵⁰, and were used as a negative control for TH expression.

TABLE 2
Equipment

| Equipment | Supplier | Part Number | Purpose |
|---|---|---|---|
| Centrifuge | Eppendorf | 5424R | Cell lysate centrifugation |
| Centrifuge | Sorvall | ST8 | Cell culture |
| Magnet | Biolegend | 480019 | Magnetic Isolation |
| Plate reader | Biorad | iMark | WB/ELISA |
| Odyssey | Licor | Odyssey | WB |
| ChemiDoc+ | Biorad | ChemiDoc MP | WB |
| Mini-Protean | Biorad | 1658005 | WB |
| Tetra | | | |
| ELISA shaker | VWR | | ELISA incubations |
| Plate Washer | BioTek | ELX-405 | ELISA washes |
| Spectral Analyzer | Sony | SP6800 | FC |

### PBMC Isolation

PBMCs express TH^{43,51}. As previously published⁵¹, whole blood was collected in K2EDTA vacutainer blood collection tubes (BD, 366643) and held at room temperature for up to 2 hours prior to PBMC isolation. Briefly, blood from healthy volunteers and PD patients was overlaid in Leucosep tubes (Table 2) for PBMC isolation, centrifuged for 20 minutes at 400 g with brakes turned off and acceleration set to minimum. PBMCs were collected from the interphase of Ficoll and PBS, transferred to a fresh 15 ml conical tube, resuspended in 8 mL sterile PBS and centrifuged for 10 minutes at 100 g, and repeated twice more. Cells were counted with a hemacytometer using trypan blue exclusion of dead cells, and density-adjusted for downstream applications.

### Magnetic Monocyte Isolation

PBMCs are composed of multiple cell subsets⁵², each with distinct function and catecholamine sensitivity^{53,54}—for example, lymphocyte regulation by catecholamines dopamine and NOR^{5,6,55} have been studied for several decades^{8,18,56,57}, while data regarding catecholamine function in myeloid lineage cells including monocytes is less abundant. In this study, we were narrowly focused on studying peripheral monocytes which we and others have previously shown to express TH^{9,51,58-60}. Because PBMCs comprise a variety of immune cell types, we used immunomagnetic enrichment to obtain a greater than 95% CD14+ monocytes that were utilized in assays described in the current study.

CD14+ monocytes express TH⁵¹. Primary CD14+ monocytes were isolated using Biolegend MojoSort magnetic isolation kit (Biolegend, 480094) per manufacturer's instructions. Briefly, 20 million total PBMCs were counted, density adjusted to 1 million cells/uL, resuspended in MojoSort buffer, and incubated with TruStain Fc-block for 10 minutes at room temperature, followed by 1:10 anti-CD14 magnetic nanobeads for 15 minutes on ice. Following 2 washes with 2.5 mL ice-cold MojoSort buffer, cell pellet was resuspended in 2.5 mL MojoSort buffer and subject to three rounds of magnetic isolation per manufacturer's instructions. The resulting cell pellet was washed to remove remaining non-CD14+ cells and subject to cell lysis as detailed below.

### Preparation of Cell Lysates

Adherent cells in culture were lifted using 0.02% EDTA in PBS, diluted with 5 volumes of PBS, and centrifuged at 100×g. Non-adherent cells (PC12) were centrifuged at 100×g for 5 minutes at room temperature, and cell pellets were washed 3 times with 5 volumes of sterile PBS. Primary macrophages and primary murine neuron cultures were washed thrice with ice-cold PBS, on ice. Cell pellets and adherent primary cells were then lysed in ice-cold lysis buffer (10 mM NaCl, 10% glycerol (v/v), 1 mM EDTA, 1 mM EGTA, and HEPES 20 mM, pH 7.6), with Triton X-100 added to a final concentration of 1%, containing 1× protease inhibitor cocktail (Millipore-Sigma, 539131) for one hour at 4° C. with rotation. Resulting lysate was centrifuged at 12,000×g for 15 minutes at 4° C. Supernatant was set aside for protein quantification by Lowry assay (Biorad, 5000112) and the remainder was stored at −80° C. until use for downstream assays.

### Western Blot

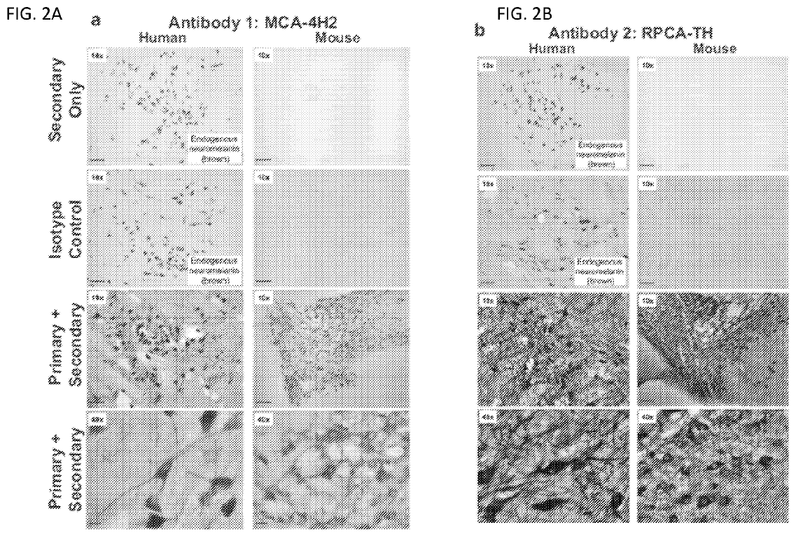

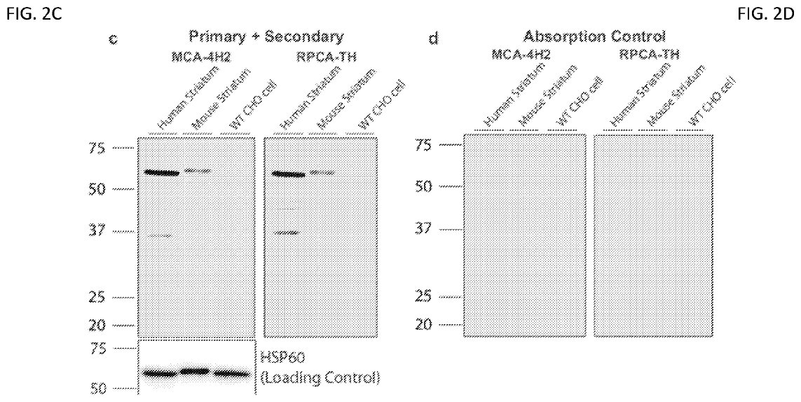

Reagents, antibodies and equipment are outlined in Tables 2, 3 and 4. Samples of PC12 lysate (5 ug) and recombinant TH protein (120 ng, 60 ng, 30 ng, 15 ng, 7.5 ng, 3.75 ng, and 1.875 ng) were incubated in Laemmli sample buffer containing 10% beta-mercaptoethanol at 37° C. for 30 minutes, separated by SDS-PAGE on 10% bis/polyacrylamide gels, and transferred to nitrocellulose membranes. After first blocking for 1 hour in TBS-T (50 mM Tris-HCl, 150 mM NaCl, and 0.1% Tween 20) containing 5% dry milk (blocking buffer), then incubated with primary antibody against TH (Table 4) overnight at 4° C. Membranes were then incubated with an appropriate secondary antibody (Table 4) for 1 hour at room temperature with agitation. Following all antibody steps, membranes were washed three times for 5 minutes each using TBS-T. TH was visualized using the Licor Odyssey (Table 2). Absorption controls were performed as followed: the primary antibodies were pre-incubated with 20 ug/mL recombinant TH protein for 30 minutes on ice, then were used to confirm primary antibody specificity (Table 3, FIG. 2C-D).

TABLE 3
Reagents and Materials

| Reagent | Supplier | Catalog Number | Purpose | Concentration | |
|---|---|---|---|---|---|
| EZ-Link Sulfo-NHS- | Thermo | A39257 | RPCA-TH biotinylation | 20-fold molar | |
| LC-Biotin | Scientific | | | excess | |
| Fat-free milk | Carnation | N/A | WB/ELISA | 1% or 5% | |
| Clarity Western | BioRad | 1705061 | WB ECL | N/A | |
| TMB Substrate | ThermoFisher | 34028 | ELISA | Stock | |
| NiDAB | Vector Labs | SK-4100 | IHC | Stock | |
| TPA | Biolegend | 755802 | ELISA, FC | 100 | ng/mL |
| IL6 | Biolegend | 570802 | ELISA, FC | 17 | ng/mL |
| TNF-alpha | Biolegend | 570102 | ELISA, FC | 17 | ng/mL |
| H2SO4 | Sigma | 339741 | ELISA | 2N | |
| TritonX-100 | ThermoFisher | BP151-100 | Magnetic Isolation | 1% | |
| Tween-20 | ThermoFisher | MP1Tween201 | TBS-T | 0.2% | |
| Protease Inhibitor | Millipore | 539191 | Cell lysis | 1x | |
| DC Protein assay | Biorad | 5000112 | Protein assay | N/A | |
| FBS | Gemini | 100-106 | Cell Culture | 10% or 5% | |
| Horse Serum | Sigma | H1138-500ML | Cell Culture | 5% | |
| Pen/Strep | ThermoFisher | 15-140-122 | Cell Culture | 1% | |
| RPMI | Corning | 10-017 | Cell Culture | Stock | |
| Immulon 4HBX | ThermoFisher | 3855 | ELISA | N/A | |
| MojoSort Buffer 5x | Biolegend | 480017 | Magnetic cell isolation | 1x | |
| Leucosep Tubes | Grenier BioOne | 227290P | PBMC isolation | N/A | |
| Ultra-low-adherence | Corning | 3471 | TPA/TNF-alpha stimulation | N/A | |
| 6-well plates | | | in vitro, for ELISA/FC | | |
| Accumax | Innovative Cell | AM105 | Cell detachment | Stock | |
| | Tech | | | | |
| CountBright beads | Invitrogen | C36950 | FC | 5000 | beads/uL |
| XPro1595 | N/A | N/A | In vitro treatment | 50 | ng/mL |

### Immunohistochemistry

Human tissues were sectioned at 40 μm on a vibrating microtome and subjected to antigen retrieval in citrate buffer (10 mM citric acid, 2 mM EDTA, 2% Tween-20, pH 6.2) at 96° C. for 30 minutes, and then allowed to cool to room temperature. PFA-perfused mouse brain tissues were also sectioned at 40 μm on a vibrating microtome.

Human and murine brain tissues were quenched for 20 minutes with 3% hydrogen peroxide, blocked and permeabilized at 37° C. for 1 hour in PBS containing 5% normal goat serum and 0.5% TritonX-100. Primary antibodies RPCA-TH and MCA-4H2 (1:500 and 1:100 dilution, respectively, Table 4) were incubated overnight, followed by secondaries conjugated to HRP (1:250, Table 4), incubated for 1 hour at room temperature. Isotype control antibodies (Biolegend, Table 1) were used to confirm specificity of RPCA-TH and MCA-4H2. Sections were detected with HRP-substrate NiDAB (Vector Labs, Table 3).

TABLE 4
Antibodies

| | | | | Catalog | | | | |
|---|---|---|---|---|---|---|---|---|
| Specificity | Clone/Species | Conjugate | Vendor | Number | Purpose | Dilution | Concentration | |
| TH | Polyclonal/Rabbit | N/A | Sigma | AB152 | WB | 1:1,000 | 0.1 | ug/mL |
| TH | Monoclonal/Mouse | N/A | EnCor | MCA-4H2 | WB, ELISA, | 1:1,000, | 1 | ug/mL |
| | | | | | IHC | 1:100 | | |
| TH | Polyclonal/Rabbit | N/A | EnCor | RPCA-TH | WB, IHC | 1:1,000, | 1 | ug/mL |
| | | | | | | 1:500 | | |
| TH | Polyclonal/Rabbit | Biotin | EnCor | RPCA-TH | ELISA | 1:6,000 | 1.65 | ug/mL |
| Chicken | Polyclonal/Rabbit | HRP | Sigma | A9046 | WB | 1:1,000 | 1 | ug/mL |
| Mouse | Polyclonal/Goat | IR-800 | Licor | 92632210 | WB | 1:15,000 | 0.0003 | ug/mL |
| Rabbit | Polyclonal/Goat | IR-680 | Licor | 92568071 | WB | 1:15,000 | 0.0003 | ug/mL |
| CD14 | Nanobeads | Magnetic | Biolegend | 480093 | Magnetic | 20 uL/20M | N/A | |
| | | | | | Isolation | cells | | |
| Biotin | N/A (Avidin) | HRP | Vector Labs | A2004 | ELISA | 1:2,500 | 0.0004 | ug/mL |
| Mouse | Polyclonal/Goat | HRP | Biolegend | 405306 | IHC | 1:250 | 0.002 | ug/mL |
| Isotype Ctl | IgG1, k/Mouse | N/A | Biolegend | 401401 | IHC, WB | 1:250-1000 | 1 | ug/mL |
| Isotype Ctl | Polyclonal/Rabbit | N/A | Biolegend | 910801 | IHC, WB | 1:100-1000 | 1 | ug/mL |
| Rabbit | Polyclonal/Donkey | HRP | Biolegend | 406401 | IHC | 1:500-1000 | 0.002 | ug/mL |
| CD14 | Polyclonal | Magnetic | Biolegend | 480048 | ELISA, FC | Mfg Instr. | N/A | |
| Ki67 | Polyclonal/Chicken | N/A | Encor | CPCA- | FC | 1:100 | 0.1 | ug/mL |
| | | | | Ki67 | | | | |
| Chicken | Polyclonal/Goat | Alexa 488 | LifeTech | A-11039 | FC | 1:100 | 0.2 | ug/mL |
| CD14 | IgG2b/Mouse | FITC | BD | M-Phi-09 | FC | 1:50 | 0.02 | ug/mL |

### Detection Antibody (RPCA-TH) Biotinylation

EZ-Link Sulfo-NHS-LC-Biotin (A39257, Thermo Scientific) at 20-fold molar biotin was used according to the manufacturer's protocol. Anti-biotin antibody was concentrated to 2 mg/mL, pH was adjusted to 8.0 at room temperature. The conjugate was purified by gel filtration on a Biorad 10DG column (cat 732-2010) at room temperature.

### ELISA for TH

Antibodies used for ELISA are described in Table 4. Ten lanes of an Immulon 4 HBX High-Binding 96 well plate were coated with 100 uL per well of 1:1,000 dilution of 1 mg/mL mouse anti-TH (MCA-4H2) in coating buffer (28.3 mM Na₂CO₃, 71.42 mM NaHCO₃, pH 9.6) for 20 hours at 4° C. Edge lanes 1 and 12 were left empty. Wells were blocked with 5% fat free milk in 1×TBS (pH 7.4) for 1 hour at room temperature on an orbital shaker set to 90 rpm. To produce a standard curve, two standard curve lanes were generated, with six serial dilutions, beginning at 10 ng/ml and 1 ng/ml in TBS-T containing 1% fat free milk (with the last well in each standard curve lane left with incubation buffer only as a blank. Remaining wells were incubated in duplicate with 100 microliters of lysates from 1.5 million cells of interest. Incubation was completed for 20 hours at 4° C. on an ELISA shaker set to 475 rpm.

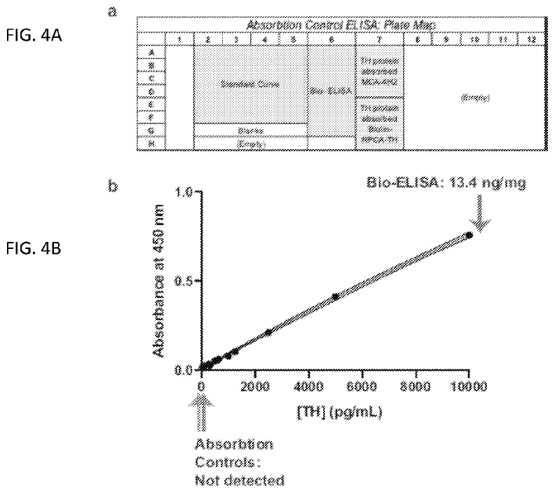

After each well was washed and aspirated 6 times with TBS-T, affinity purified polyclonal rabbit anti-TH (EnCor, RPCA-TH) conjugated to biotin was diluted 1:6,000 from a stock concentration of 1.65 mg/mL in TBS-T with 1% fat-free milk and incubated for 1 hour at room temperature at 425 rpm. 100 uL Avidin-HRP (Vector labs, A-2004), diluted 1:2,500 in TBS-T with 1% fat-free milk, was added to each well following washing as described above, and incubated for 1 hour at room temperature at 425 rpm. Following final washes, 150 uL room temperature TMB-ELISA reagent (Thermo Fisher, 34028) was added to each well. The reaction was allowed to continue for 20 minutes, protected from light, and stopped by addition of 50 uL 2N H₂SO₄. The plate was immediately read at 450 nm. Absorption controls (FIG. 4) were conducted by pre-incubating MCA-4H2 and RPCA-TH with a 20-fold excess concentration of recombinant TH protein for 30 minutes on ice, prior to addition to the ELISA plate, followed by the remainder of the protocol described above.

Duplicate standard and sample wells were averaged, and background-subtracted based on blank wells. The concentration of TH for each experimental group was calculated using a quadratic curve equation calculated in Graphpad Prism 8, then normalized to total protein concentration per sample as calculated using the Lowry assay. Samples which produced negative values for TH concentration were considered below detection threshold, and therefore assigned a value of 0. Final TH values shown are presented as pg TH/mg total protein after multiplication of the nanogram TH value by 1,000 to show TH as picogram TH/milligram total protein.

In Vitro Stimulation/Treatment with TNFα, Tissue Plasminogen Activator (TPA), TNFα Inhibitor XPro1595 and IL6

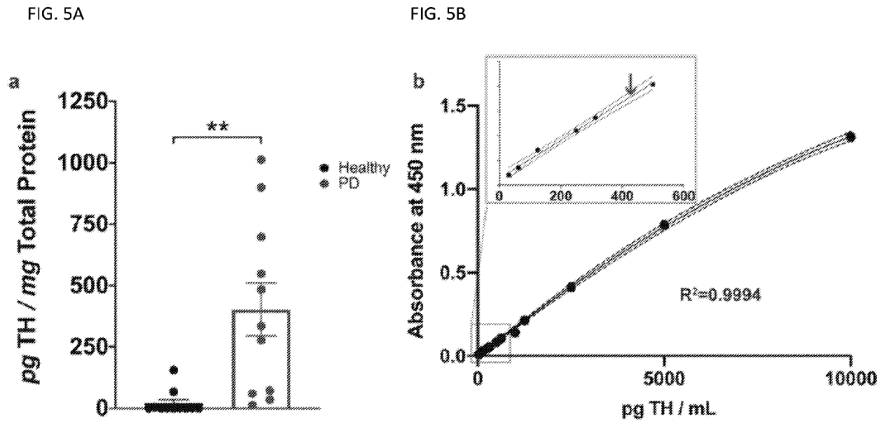

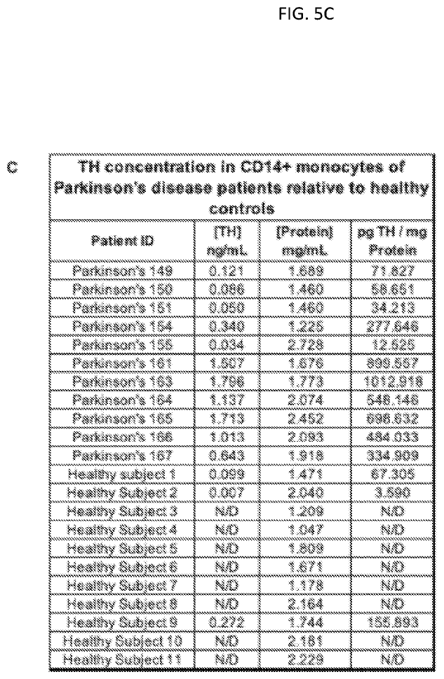

Monocytes were isolated from total PBMCs prepared as described above⁵¹ using negative selection (Biolegend, 480048) per manufacturer's instructions. Total PBMCs were Fc-blocked to reduce nonspecific binding, followed by incubations with biotin-conjugated antibody cocktail containing antibodies against all subsets except CD14 (negative selection), followed by incubation with magnetic-Avidin beads, allowing all subsets other than CD14+ monocytes to be bound to the magnet. Monocyte purity/enrichment was routinely verified to confirm that the final cell population was greater than 95% pure CD14+ cells (FIG. S2). CD14+ monocytes were collected from the supernatant fraction, washed, counted and density adjusted such that 2 million CD14+ monocytes were seeded per well (FIG. 5A) and treated for 4 hours with vehicle, TPA (100 ng/ml, Biolegend, 755802)⁷, TNFα (17 ng/mL, Biolegend, 570102)⁶¹, XPro1595 (50 ng/ml), or IL6 (17 ng/mL) in an ultra-low-adherence 6-well plate (Corning, 3471) to prevent adherence. Suspended cells from each treatment group were aspirated and placed in a 15 ml conical tube, with any remaining adherent cells detached by incubation with 700 uL Accumax solution for 3 minutes (Innovative Cell Technologies, AM105) and added to suspended cells. After pelleting cells by centrifugation (3 minutes×100 g, room temperature), cells were assayed by either flow cytometry⁵¹ or lysed for ELISA as stated above (“Preparation of cell lysates”).

As previously published⁵¹, cells for flow cytometry were fixed and permeabilized (eBioscience, 88-8824-00), and stained for intracellular marker TH (Millipore-Sigma, AB152, 1:100) followed by a species-specific secondary (anti-Rabbit BV421, BD, 565014). After resuspending the sample in a final volume of 250 uL PBS, 5 uL of Invitrogen CountBright Absolute Counting Beads (5000 beads/mL, Invitrogen, C36950) were added just prior to data acquisition (Sony Spectral Analyzer, SP6800). Monocytes were gated for single cells and positive TH expression (FIG. 5B), and normalized to counting beads in each sample to obtain an absolute count of TH+ monocytes per uL suspension.

### Statistics

A two-tailed, unpaired T test was used to compare TH quantity in PD patients versus healthy control. In this experiment, P<0.05 was considered statistically significant. One-way ANOVA with Tukey's correction for multiple comparisons was used to compare TH-expressing monocytes assayed by flow cytometry and ELISA following treatment with TPA, TNFα, XPro1595, IL6 or Vehicle. P<0.05 was considered statistically significant.

### Example 2: Bio-ELISA Successfully and Reproducibly Detects Recombinant and Native TH

To test the hypothesis that, similar to CNS in PD, TH expression is reduced in peripheral blood monocytes, a sensitive assay needed to be developed to quantify TH levels in monocytes from healthy controls, as well as various reference TH expressing systems. Given the plethora of biological systems expressing TH, there is an unmet need for a sensitive and reliable assay to quantify TH levels which with broad biological implications in basic science, preclinical and clinical research. To date, measurement of TH levels in midbrain neurons has been accomplished by immunohistochemistry, and Western blot⁶²⁻⁶⁵, while TH levels in peripheral immune cells has been assayed by flow cytometry⁵¹. Although reliable, these methods share a common shortcoming in that they are semi-quantitative at best, and at worst only indicate the presence or absence of TH. Accordingly, presented herein is an embodiment directed to a highly sensitive and fully quantitative enzyme-linked immunosorbent assay (Bio-ELISA) to measure TH protein levels.

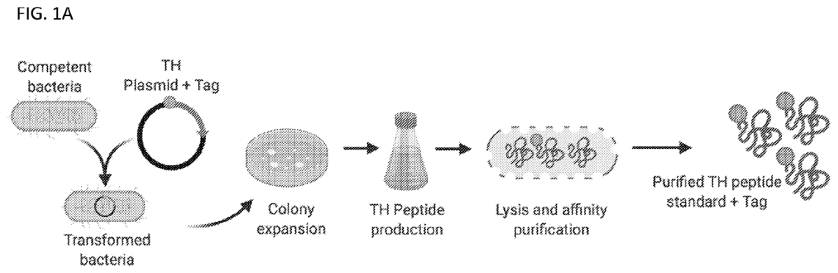

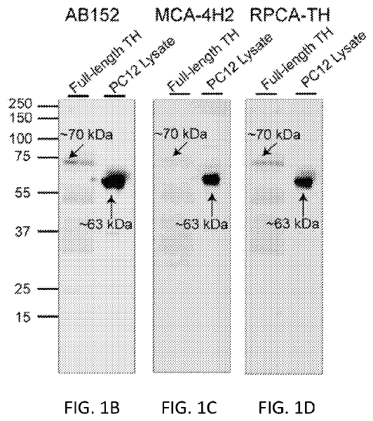

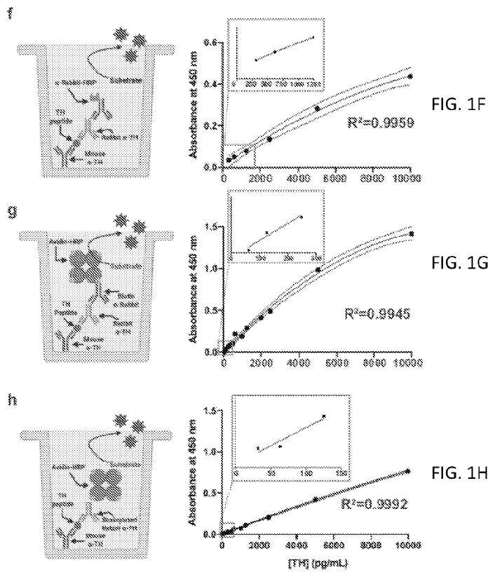

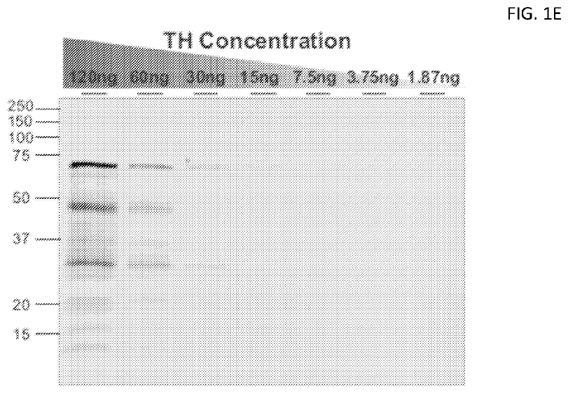

Quantification of TH using Bio-ELISA depends on the availability of purified TH and high-quality antibodies against TH, preferably generated in two distinct host species. A panel of monoclonal and polyclonal antibodies were generated against full length recombinant human TH (FIG. 1A), and quality assessment was performed by standard ELISA, Western blotting, and appropriate cell and tissue staining. These novel antibodies behaved in all respects similarly to a widely used commercial TH antibody (FIG. 1B, AB152, Millipore-Sigma)⁶⁶⁻⁶⁹. A mouse monoclonal antibody, MCA-4H2, and a rabbit polyclonal, RPCA-TH, were selected as ELISA capture and detection antibodies, respectively.

Next, TH recombinant protein band identity was compared to TH expression in PC12 cells (FIG. 1C). As predicted, PC12 lysate shows a single TH band at ˜63 kDa, with a corresponding band for the TH recombinant protein at ˜70 kDa. The observed difference in molecular weight between TH expressed in PC12 cells and recombinant TH protein is due to the additional 5.7 kDa N-terminal His-tag. Lower molecular weight bands (at 50 kDa and 35 kDA, FIG. 1B-E) represent proteolytic cleavage products of mammalian TH when expressed in a prokaryotic system. Both MCA-4H2 and RPCA-TH reliably detect both recombinant TH and native TH in PC12 lysates (FIG. 1 C, D).

Since antibody specificity is crucial for developing a novel assay, specificity was rigorously confirmed. First, MCA-4H2 and RPCA-TH were used to stain human and murine midbrain tissue (FIG. 2). MCA-4H2 (FIG. 2A) and RPCA-TH (FIG. 2B) both showed high specificity for TH+ dopamine neurons in both human and murine tissues with no visible background. In addition, both secondary-only and isotype control staining show minimal background (FIG. 2A-B, top and second panels). Lastly, both MCA-4H2 and RPCA-TH were tested via Western blot using standard immunoblotting as well as blocking peptide/absorption controls (FIG. 2C-D). Both antibodies show good specificity and minimal background. CHO cells, used as the negative control since they do not express TH, show no TH band (FIG. 2C). The peptide blocking/absorption control groups (FIG. 2D) also show no detectable signal, further confirming specificity.

Next, 1:1 serial dilutions of TH recombinant protein were prepared in Laemmli buffer, from 6 ug/mL to 0.094 ug/mL, to test the limits of detection using the Licor IR imaging system for Western blot (FIG. 1E) using commercially available TH antibody AB152. While effective, detection via Licor Odyssey using an IR fluorescent dye affords a fixed lower detection limit of ˜15 ng, suggesting that IR fluorescent imaging is suitable for high expressing systems, but unsuitable for accurate quantification at low nanogram or picogram TH levels, reinforcing the need for a more sensitive, quantitative TH Bio-ELISA.

To quantify TH expression in control conditions, a standard sandwich ELISA approach (FIG. 1F) was first attempted, in which MCA-4H2 was used as the capture antibody, followed by incubation with recombinant TH, then RPCA-TH as detection antibody. Enzyme-based detection was accomplished by addition of HRP-conjugated secondary (goat anti-rabbit HRP, Vector, BA1000). While this reliably quantified TH, the standard version of this assay produced a lower detection threshold of 125 pg/mL TH. A further increase in sensitivity of the assay was sought by addition of a biotin-avidin amplification step (Avidin-HRP, Vector, A2004) (FIG. 1G), which provided an improved lower threshold of 62.5 pg/mL. A further refinement was the biotinylation of the rabbit detection antibody using Sulfo-NHS-LC-biotin (Thermo Scientific A39257) which improved sensitivity further by reducing background and producing a lower-threshold of detection at 15 pg/mL (FIG. 1H) with biotinylated antibodies, hence the Bio-ELISA designation. It was found that the Bio-ELISA is around one thousand-fold more sensitive than infrared Western blot imaging (15 pg/mL vs. 15 ng/ml). Both TH antibodies are available commercially from EnCor Biotechnology Inc. According to another embodiment, the invention is directed to a method that includes the method steps as depicted in FIG. 1G or 1H.

### Example 3: Antibodies MCA-4H2 and RPCA-TH Reliably Detect Both Native and Denatured TH in Mouse and Human Tissue

Aiming to develop a novel and reliable ELISA for both human and murine tissues, there was a need to confirm specificity of these antibodies on native and denatured tissues from both human and mouse brain regions rich in tyrosine hydroxylase (FIG. 2). MCA-4H2 (FIG. 2A) and RPCA-TH (FIG. 2B) detect TH+ cell bodies and neuronal processes in both human and mouse midbrain. Minimal non-specific staining detected in secondary only and isotype controls, further confirming antibody specificity. Similarly, both MCA-4H2 and RPCA-TH detect denatured TH on Western blot (FIG. 2C) following separation on SDS-PAGE, with minimal non-specific bands in the negative control (parental CHO cell homogenate). HSP60 is shown as a loading control. As an additional validation step to confirm the specificity of MCA-4H2 and RPCA-TH, primary antibodies were pre-incubated with recombinant TH protein (blocking peptide/absorption control) and show no observable signal (FIG. 2D).

### Example 4: TH Bio-ELISA Reliably Quantifies TH in PC12 Cells, Human Macrophages, and Cultured Murine Dopamine Neurons

Having established a reliable method with a suitably low detection threshold, the TH Bio-ELISA was tested on cell homogenates prepared from PC12 cells, HEK293 cells, cultured primary human macrophages derived from whole blood samples from healthy donors, and primary cultures of midbrain dopamine neurons prepared from PND0-PND3 mouse pups. PC12 cells are known to express high levels of TH⁴⁸, while HEK293 serve as negative control^{45,70}. Cultured midbrain dopamine neurons are known to express TH as the rate limiting enzyme for dopamine⁴⁷ while cultured human monocyte-derived-macrophages express TH protein and mRNA^{9,43}.

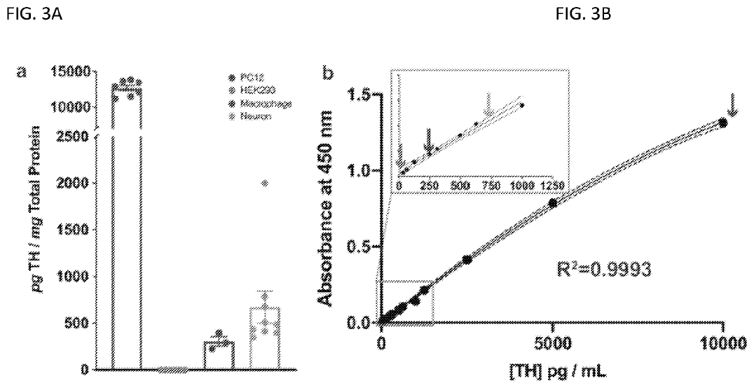

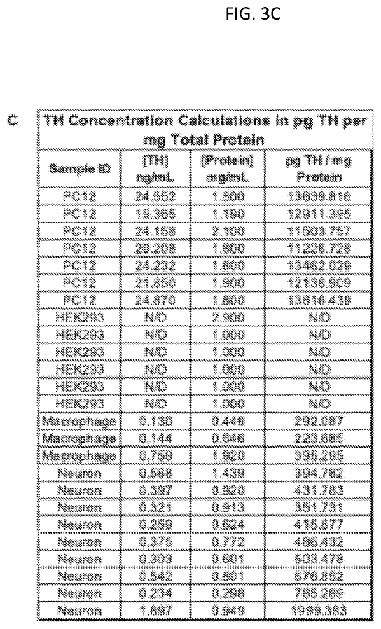

TH expression is shown as unit TH (picogram or nanogram) per mg total protein, as determined by the Lowry assay. PC12 homogenate provided a reliable positive control expressing high levels of TH (>10 ng TH/mg total protein), while HEK293 homogenate showed no detectable levels of TH, in at least 6 independent replicates. As anticipated, cultured dopamine neurons from postnatal mice showed greater TH concentrations (˜700 pg TH/mg total protein) than cultured human macrophages (˜300 pg TH/mg total protein) (FIG. 3A), suggesting the Bio-ELISA is applicable to cell and tissue samples derived from human and murine specimens, paving the way for its application in translational and preclinical studies involving measurements of TH protein. It is noted that unlike cultured human monocyte-derived-macrophages, cultured dopamine neurons contain various cell types, and consist of 12-16% dopamine neurons. The remainder are GABAergic neurons and supporting cells (microglia and astroglia)⁷¹⁻⁷³. Thus, it is believed that TH levels are much higher in a single dopamine neuron than in a macrophage. Visual representation of relative TH expression in PC12, HEK293, human macrophage and primary neuron homogenates are plotted on a representative standard curve (FIG. 3B). Raw values [TH] in ng/mL calculated from absorbance are shown in FIG. 3C, alongside each sample ID. Raw TH concentration was divided by [Protein], then multiplied by 1,000 to produce values in pg TH/mg total protein (FIG. 3C).

To further confirm the specificity of these antibodies, the Bio-ELISA was tested using absorption controls (FIG. 4). In multiple independent replicates, a single ELISA plate was prepared as shown in FIG. 4A (Bio-ELISA, blue; absorbed MCA-4H2, orange; absorbed RPCA-TH, green), and incubated with PC12 lysate as a positive control. Following peptide blocking/absorption of either capture or detection antibody, PC12 cell lysate yields no detectable TH (orange and green arrows, FIG. 4B), while the TH Bio-ELISA (blue arrow) recapitulates TH concentrations measured in PC12 cells (compare FIG. 3 panel A-B with FIG. 4 panel B).

### Example 5: Monocytes Isolated from Blood of PD Patients Show Increased TH Protein Relative to Age-Matched Healthy Controls

PD is a disease in which monoamine signaling is affected in both CNS and peripheral immune cells⁹. The literature supports the hypothesis that similar to the CNS, peripheral TH expression is altered, but there is no reliable information about the direction of this change. Since peripheral immune cells including PBMCs express the machinery for catecholamine synthesis, including TH, they provide a biologically relevant peripheral tissue preparation to investigate TH levels in monocytes of PD patients and age-matched healthy subjects. Monocytes for each subject were isolated from 20 million total peripheral blood mononuclear cells (PBMCs) using anti-CD14 magnetic isolation per manufacturer's instructions. Purified monocytes were immediately lysed and assayed via Bio-ELISA for TH concentration following total protein quantification. Of 11 healthy control samples included, only three registered TH concentrations above the detection threshold. By contrast, all 11 PD patients recruited for this study show clear positive TH values that were significantly higher than healthy controls. These data suggest that, contrary to our original hypothesis, PD monocytes express significantly more TH protein relative to healthy control subjects (FIG. 5A—n=11, t[20]=3.777, P=0.0012). Mean TH protein concentrations in PD monocytes are shown on a representative standard curve (FIG. 5B), along with raw data used to calculate TH concentrations (FIG. 5C). While these data represent a snapshot of TH levels in circulation PD monocytes, we cannot make any overarching claims that TH levels in monocytes precede clinical symptoms of PD, or predict a PD diagnosis. A larger sample numbers and longitudinal studies can test these possibilities. Nevertheless, these data suggest that in peripheral monocytes of Parkinson's patients, the rate limiting protein involved in catecholamines synthesis is increased. Investigating the potential mechanism was the focus of the next set of experiments.

### Example 6: TNFα Increases Number of TH+ Monocytes, and the Amount of TH Protein Per Monocyte

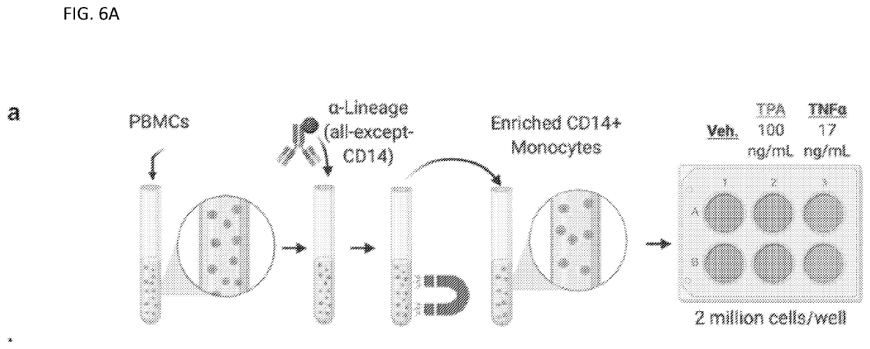

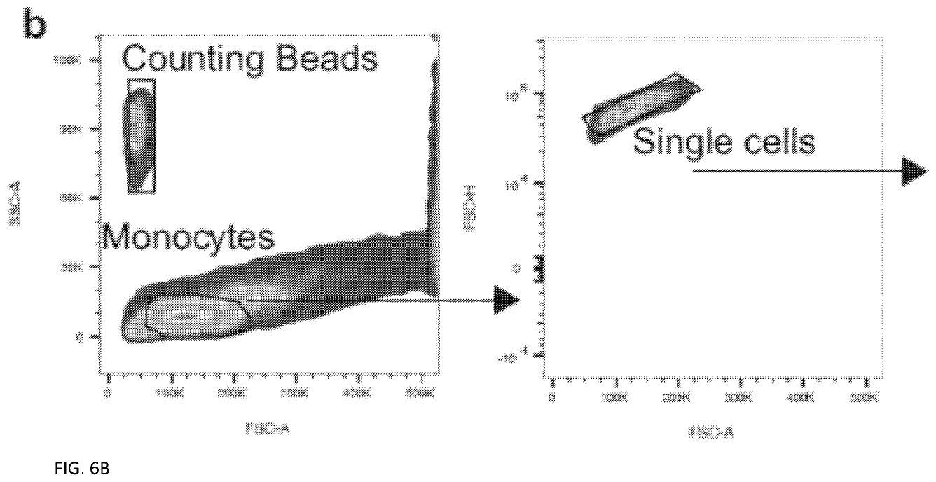

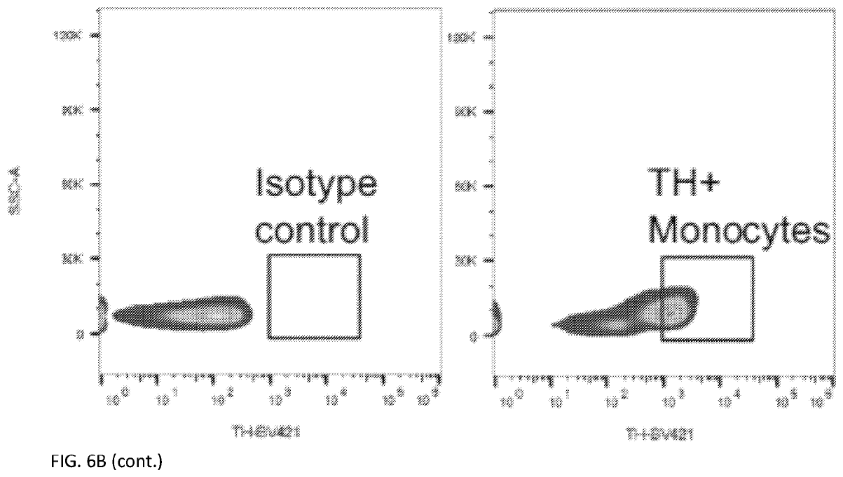

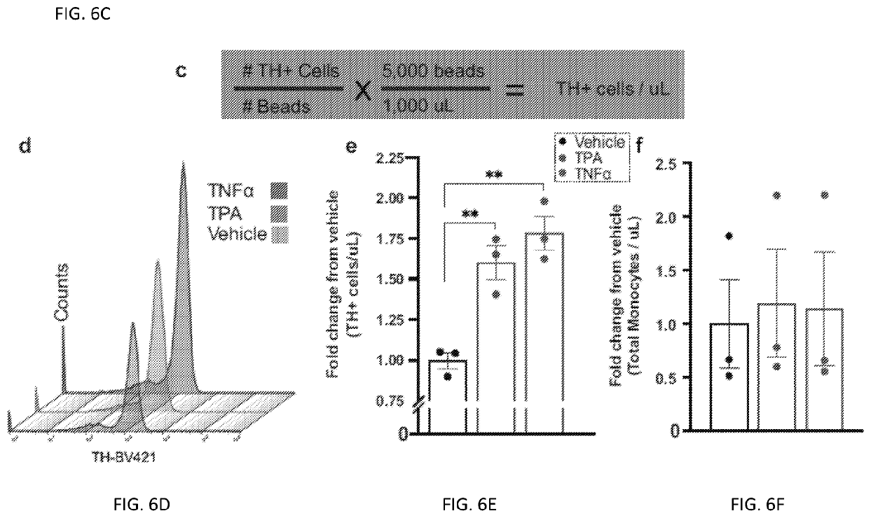

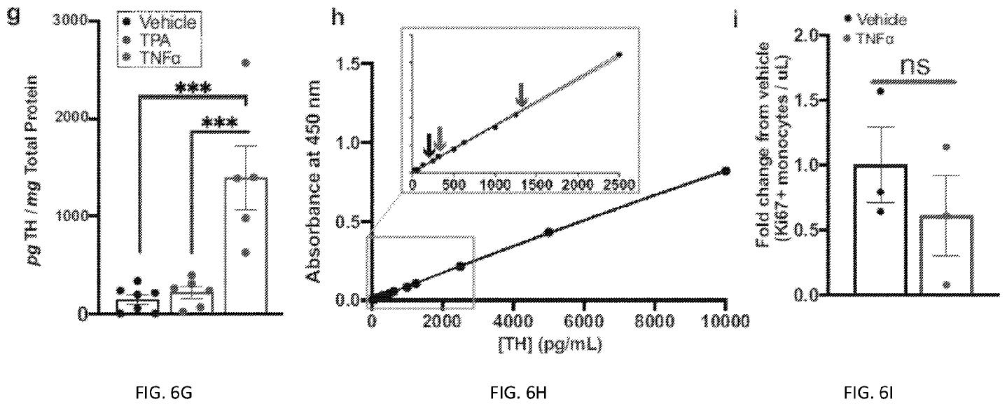

There is strong evidence in the literature for increased TNFα in PD^{27,34-36} including in the brain, cerebrospinal fluid, and serum of Parkinson's patients²⁷ as well as in Parkinsonian mice^{37,38}. These reports suggest that TNFα plays a role in the often hypothesized peripheral inflammation in PD⁷⁴⁻⁷⁹, which is also documented in other inflammatory states including rheumatoid arthritis^{80,81} and multiple sclerosis⁷, where TH expression is linked to TNFα expression^{80,81,7}. Therefore, the hypothesis that ex vivo stimulation of monocytes from healthy subjects with TNFα stimulates TH expression was tested, as measured by changes in the number of TH-expressing monocytes, and/or the amount of TH per monocyte. Flow cytometry was employed to address the former, and bio-ELISA to address the latter. Two million monocytes isolated from whole blood of healthy donors were treated for 4 hours with tissue plasminogen activator (TPA, 100 ng/ml, positive control for increased monocyte TH expression⁷), TNFα (17 ng/mL)⁶¹ and compared with monocytes treated with vehicle (FIG. 6A). Monocytes were assayed for TH expression by two complementary methods: flow cytometry⁵¹ (FIG. 6B-F) and ELISA (FIG. 6G-H). It is noted that because a prolonged TNFα exposure can induce cell toxicity⁸²⁻⁸⁶, multiple treatment durations were tested. It was found that a 4-hour TNFα (17 ng/ml)⁶¹ treatment had a minimal effect on cell viability; whereas, a longer TNFα exposure substantially decreased cell viability. Therefore, a 4-hour treatment strategy was selected in this study.

To control for donor variability, identical quantities of counting beads were added as a reference. The number of TH+ monocytes was quantified by flow cytometry (FIG. 6B, left) in two experimental groups: TPA-treated and TNFα-treated. Monocytes in each condition were gated to isolate single cells expressing TH (FIG. 6B). Raw counts of monocytes in each condition revealed increased monocytes expressing TH after treatment with TPA or TNFα (FIG. 6D), while the number of TH+ monocytes per microliter (FIG. 6C) are significantly increased relative to vehicle (FIG. 6E; N=3, F(2,6)=0.364, p=0.0018), suggesting that the number of TH+ monocytes increase following treatment with TNFα or positive TPA control. A possible mechanism for this observation is either monocyte proliferation during the treatment period or an altered monocyte phenotype in response to TNFα, with no change in total number of monocytes. In other words, following TNFα treatment, TH+ monocytes may either be increasing in number (proliferation) or existing monocytes upregulate TH expression and become TH+ (phenotypic change). While a four-hour exposure to TNFα is an insufficient time period to induce proliferative events in immune cells⁷⁴, these possibilities could not be confidently ruled out without additional analyses. Therefore, monocyte proliferation was quantified by comparing the total number of monocytes per microliter of untreated vs. TNFα treated experimental group. It was found that the total number of monocytes per microliter to be unchanged (FIG. 6F). While these results suggest that monocyte proliferation did not occur in response to TNFα, the results of simple cell counts are not definitive. A more rigorous approach was conducted to assess Ki67 expression as a measure of cell proliferation⁸⁷. Ki67 expression in TNFα treated monocytes relative to vehicle-treated controls revealed no change in Ki67 expression following TNFα treatment (FIG. 6I). Thus, these results showed phenotypic changes in monocytes, but not cell proliferation in response to TNFα. While this finding explains our earlier observation of increased numbers of TH+ cells, the potential phenotypic shift following TNFα mediated immune stimulation was an unpredicted and novel finding.

The flow cytometry data strongly support the conclusion that TNFα increases numbers of TH+ monocytes, but increased number of TH+ monocytes could be due to increased numbers of cells expressing TH protein, increased quantity of TH protein per cell, or both. In order to determine whether or not TNFα treatment increases quantity of TH protein per monocyte, identically treated monocytes were lysed and assayed using our TH Bio-ELISA. It was found that four-hour treatments with TNFα significantly increased the amount of TH protein (picogram TH per milligram total protein) above both vehicle and the positive control group (TPA treatment; FIG. 6G; n=5-6 per group, F(2,15)=3.297, p=0.0001), indicating that exposure to TNFα is sufficient to increase TH protein in human monocytes. Overall, the data show that TNFα increases both the number of monocytes expressing TH and the quantity of TH expressed by each cell.

### Example 7: Inhibition of TNFα Blocks Increase in Number of TH+ Monocytes and Amount of TH Per Monocyte

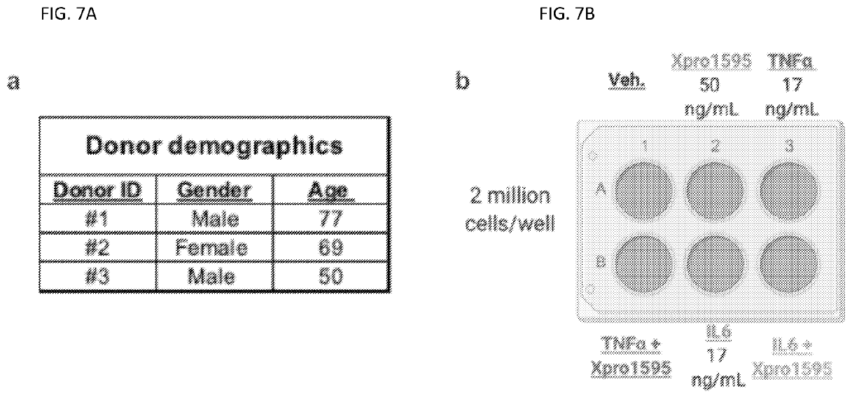

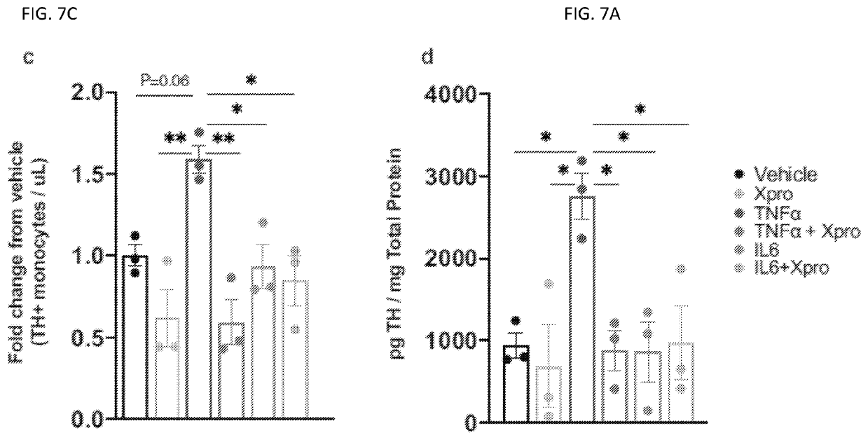

To determine the specificity of TNFα regulation of TH in monocytes, two approaches were employed. It was investigated whether inhibition of TNFα signaling attenuates or blocks the TNFα mediated increase in TH. In addition, it was investigated whether or not interleukin-6 (IL6), a cytokine with pleiotropic effects⁷¹ that is also increased in PD⁸⁸⁻⁹⁰, and is associated with non-motor symptoms of PD⁸⁸⁻⁹⁰ can also regulate TH expression in the peripheral monocytes. To test these possibilities, XPro1595, a TNFα inhibitor^{91,92}, was studied to see if it reduces monocyte TH expression relative to TNFα treatment alone. In parallel experiments, monocytes were treated with IL6. Two million monocytes isolated from whole blood of healthy donors (FIG. 7A) were treated with XPro1595 alone (50 ng/mL), TNFα (17 ng/ml) or TNFα plus XPro1595 (FIG. 7B), IL6 alone (17 ng/mL) or IL6 plus XPro1595. The cells were subjected to flow cytometry or Bio-ELISA. It was found relative to TNFα treatment alone, XPro1595 inhibition of TNFα reduced both the number of TH+ monocytes and the quantity of TH per monocyte (FIGS. 7C and D) 71, suggesting that soluble TNFα mediates increased TH in human monocytes. As shown in FIGS. 7C and 7D, IL6 neither changed the number of TH+ monocytes nor the quantity of TH per monocyte. It should be noted that the data show that TNFα is capable of regulating TH in monocytes whereas other elevated cytokines, including IL6, donot. Since the effect of additional, non-upregulated cytokines was not tested, it cannot be said that TH exclusively mediated by TNFα. Instead, it appears clear that TNFα is capable of regulating monocytic TH. In addition, while the direct link between increased TH protein in PD monocytes and TNFα has not been investigated, the ex vivo data (FIG. 6) supports the interpretation that TNFα plays a role in increased TH expression in immune cells of PD patients.

The above examples present a highly reproducible and quantitative Bio-ELISA to measure TH protein levels in murine and human cells. Following validation of the assay in multiple TH expression systems, TH expression in PD immune cells and of age-matched healthy control subjects investigated. It was observed that PD patients' monocytes expressed significantly greater amounts of TH per monocyte. Inspired by the literature indicating increased TNFα in PD, an intriguing link between TNFα stimulation and increased TH expression in healthy monocytes was uncovered, which is attenuated by treatment with TNFα inhibitor Xpro1595. Given that TH expression and catecholamine release has been shown to be associated with an anti-inflammatory effect and can mitigate TNFα mediated inflammation, it is posited that increased TH expression in monocytes in response to elevated TNFα is a compensatory mechanism. This observation is a step towards understanding the potential underlying mechanism and functional consequence of changes in catecholamines in peripheral immune system in PD. Whereas, TH can be quantified in a 30 mL blood sample (FIGS. 3-5), for functional assays (FIGS. 6 and 7) a large blood volume (˜500 mL) is required, which is not feasible in PD subjects.

### REFERENCES

1 Molinoff, P. B. & Axelrod, J. Biochemistry of catecholamines. Annu Rev Biochem 40, 465-500, doi: 10.1146/annurev.bi.40.070171.002341 (1971).
2 Nagatsu, T., Levitt, M. & Udenfriend, S. TYROSINE HYDROXYLASE. THE INITIAL STEP IN NOREPINEPHRINE BIOSYNTHESIS. J Biol Chem 239, 2910-2917 (1964).
3 Berod, A., Biguet, N. F., Dumas, S., Bloch, B. & Mallet, J. Modulation of tyrosine hydroxylase gene expression in the central nervous system visualized by in situ hybridization. Proc Natl Acad Sci USA 84, 1699-1703, doi: 10.1073/pnas.84.6.1699 (1987).
4 Bertler, A. & Rosengren, E. Occurrence and distribution of catechol amines in brain. Acta Physiol Scand 47, 350-361 (1959).
5 Marino, F. et al. Endogenous catecholamine synthesis, metabolism storage, and uptake in human peripheral blood mononuclear cells. Exp Hematol 27, 489-495 (1999).
6 Cosentino, M. et al. Endogenous catecholamine synthesis, metabolism, storage and uptake in human neutrophils. Life Sci 64, 975-981, doi: 10.1016/s0024-3205 (99) 00023-5 (1999).
7 Cosentino, M. et al. Catecholamine production and tyrosine hydroxylase expression in peripheral blood mononuclear cells from multiple sclerosis patients: effect of cell stimulation and possible relevance for activation-induced apoptosis. J Neuroimmunol 133, 233-240, doi: 10.1016/s0165-5728 (02) 00372-7 (2002).
8 Cosentino, M. et al. Interferon-gamma and interferon-beta affect endogenous catecholamines in human peripheral blood mononuclear cells: implications for multiple sclerosis. J Neuroimmunol 162, 112-121, doi: 10.1016/j.jneuroim.2005.01.019 (2005).
9 Matt, S. M. & Gaskill, P. J. Where Is Dopamine and how do Immune Cells See it?: Dopamine-Mediated Immune Cell Function in Health and Disease. J Neuroimmune Pharmacol, doi: 10.1007/s11481-019-09851-4 (2019).
10 Weihe, E., Depboylu, C., Schutz, B., Schäfer, M. K. & Eiden, L. E. Three types of tyrosine hydroxylase-positive CNS neurons distinguished by dopa decarboxylase and VMAT2 co-expression. Cell Mol Neurobiol 26, 659-678, doi: 10.1007/s10571-006-9053-9 (2006).
11 Harris, R. C. & Zhang, M. Z. Dopamine, the kidney, and hypertension. Curr Hypertens Rep 14, 138-143, doi: 10.1007/s11906-012-0253-z (2012).
12 Wolfovitz, E. et al. Derivation of urinary dopamine from plasma dihydroxyphenylalanine in humans. Clin Sci (Lond) 84, 549-557, doi: 10.1042/cs0840549 (1993).
13 Mohanty, P. K. et al. Myocardial norepinephrine, epinephrine and dopamine concentrations after cardiac autotransplantation in dogs. J Am Coll Cardiol 7, 419-424, doi: 10.1016/s0735-1097 (86) 80515-0 (1986).
14 Fhaner, M. J., Galligan, J. J. & Swain, G. M. Increased catecholamine secretion from single adrenal chromaffin cells in DOCA-salt hypertension is associated with potassium channel dysfunction. ACS Chem Neurosci 4, 1404-1413, doi: 10.1021/cn400115v (2013).
15 Leszczyszyn, D. J. et al. Secretion of catecholamines from individual adrenal medullary chromaffin cells. J Neurochem 56, 1855-1863, doi: 10.1111/j.1471-4159.1991.tb03441.x (1991).
16 Wightman, R. M. et al. Temporally resolved catecholamine spikes correspond to single vesicle release from individual chromaffin cells. Proc Natl Acad Sci USA 88, 10754-10758, doi: 10.1073/pnas.88.23.10754 (1991).
17 Gaskill, P. J., Carvallo, L., Eugenin, E. A. & Berman, J. W. Characterization and function of the human macrophage dopaminergic system: implications for CNS disease and drug abuse. J Neuroinflammation 9, 203, doi: 10.1186/1742-2094-9-203 (2012).
18 Cosentino, M. et al. Human CD4+CD25+ regulatory T cells selectively express tyrosine hydroxylase and contain endogenous catecholamines subserving an autocrine/paracrine inhibitory functional loop. Blood 109, 632-642, doi: 10.1182/blood-2006-01-028423 (2007).
19 Lindgren, N. et al. Regulation of tyrosine hydroxylase activity and phosphorylation at Ser (19) and Ser (40) via activation of glutamate NMDA receptors in rat striatum. J Neurochem 74, 2470-2477, doi: 10.1046/j. 1471-4159.2000.0742470.x (2000).
20 Kawahata, I. & Fukunaga, K. Degradation of Tyrosine Hydroxylase by the Ubiquitin-Proteasome System in the Pathogenesis of Parkinson's Disease and Dopa-Responsive Dystonia. Int J Mol Sci 21, doi: 10.3390/ijms21113779 (2020).
21 Congo Carbajosa, N. A. et al. Tyrosine hydroxylase is short-term regulated by the ubiquitin-proteasome system in PC12 cells and hypothalamic and brainstem neurons from spontaneously hypertensive rats: possible implications in hypertension. PLOS One 10, e0116597, doi: 10.1371/journal.pone.0116597 (2015).
22 Johnson, M. E., Salvatore, M. F., Maiolo, S. A. & Bobrovskaya, L. Tyrosine hydroxylase as a sentinel for central and peripheral tissue responses in Parkinson's progression: Evidence from clinical studies and neurotoxin models. Prog Neurobiol 165-167, 1-25, doi: 10.1016/j.pneurobio.2018.01.002 (2018).
23 Salvatore, M. F., Calipari, E. S. & Jones, S. R. Regulation of Tyrosine Hydroxylase Expression and Phosphorylation in Dopamine Transporter-Deficient Mice. ACS Chem Neurosci 7, 941-951, doi: 10.1021/acschemneuro.6b00064 (2016).
24 Wang, Y., Sung, C. C. & Chung, K. K. K. Novel enhancement mechanism of tyrosine hydroxylase enzymatic activity by nitric oxide through S-nitrosylation. Scientific Reports 7, 44154, doi: 10.1038/srep44154 (2017).
25 Daubner, S. C., Le, T. & Wang, S. Tyrosine hydroxylase and regulation of dopamine synthesis. Arch Biochem Biophys 508, 1-12, doi: 10.1016/j.abb.2010.12.017 (2011).
26 Blanchard-Fillion, B. et al. Nitration and inactivation of tyrosine hydroxylase by peroxynitrite. J Biol Chem 276, 46017-46023, doi: 10.1074/jbc.M105564200 (2001).
27 Mogi, M. et al. Tumor necrosis factor-alpha (TNF-alpha) increases both in the brain and in the cerebrospinal fluid from parkinsonian patients. Neurosci Lett 165, 208-210, doi: 10.1016/0304-3940 (94) 90746-3 (1994).
28 Hirsch, E. C. et al. The role of glial reaction and inflammation in Parkinson's disease. Ann NY Acad Sci 991, 214-228, doi: 10.1111/j.1749-6632.2003.tb07478.x (2003).
29 Harris, J. P. et al. Emerging regenerative medicine and tissue engineering strategies for Parkinson's disease. NPJ Parkinsons Dis 6, 4, doi: 10.1038/s41531-019-0105-5 (2020).
30 Foffani, G. & Obeso, J. A. A Cortical Pathogenic Theory of Parkinson's Disease. Neuron 99, 1116-1128, doi: 10.1016/j.neuron.2018.07.028 (2018).
31 Ichinose, H. et al. Quantification of mRNA of tyrosine hydroxylase and aromatic L-amino acid decarboxylase in the substantia nigra in Parkinson's disease and schizophrenia. J Neural Transm Park Dis Dement Sect 8, 149-158, doi: 10.1007/bf02250926 (1994).
32 Cosentino, M. et al. Stimulation with phytohaemagglutinin induces the synthesis of catecholamines in human peripheral blood mononuclear cells: role of protein kinase C and contribution of intracellular calcium. J Neuroimmunol 125, 125-133, doi: 10.1016/s0165-5728 (02) 00019-x (2002).
33 Mogi, M. et al. Homospecific activity (activity per enzyme protein) of tyrosine hydroxylase increases in parkinsonian brain. J Neural Transm 72, 77-82, doi: 10.1007/bf01244634 (1988).
34 Kouchaki, E. et al. Increased serum levels of TNF-alpha and decreased serum levels of IL-27 in patients with Parkinson disease and their correlation with disease severity. Clin Neurol Neurosurg 166, 76-79, doi: 10.1016/j.clineuro.2018.01.022 (2018).
35 Rathnayake, D., Chang, T. & Udagama, P. Selected serum cytokines and nitric oxide as potential multi-marker biosignature panels for Parkinson disease of varying durations: a case-control study. BMC Neurol 19, 56, doi: 10.1186/s12883-019-1286-6 (2019).
36 Qin, X. Y., Zhang, S. P., Cao, C., Loh, Y. P. & Cheng, Y. Aberrations in Peripheral Inflammatory Cytokine Levels in Parkinson Disease: A Systematic Review and Meta-analysis. JAMA Neurol 73, 1316-1324, doi: 10.1001/jamaneurol.2016.2742 (2016).
37 McCoy, M. K. & Tansey, M. G. TNF signaling inhibition in the CNS: implications for normal brain function and neurodegenerative disease. J Neuroinflammation 5, 45, doi: 10.1186/1742-2094-5-45 (2008).
38 McCoy, M. K. et al. Blocking soluble tumor necrosis factor signaling with dominant-negative tumor necrosis factor inhibitor attenuates loss of dopaminergic neurons in models of Parkinson's disease. J Neurosci 26, 9365-9375, doi: 10.1523/JNEUROSCI.1504-06.2006 (2006).
39 Lee, J. K., Tran, T. & Tansey, M. G. Neuroinflammation in Parkinson's disease. J Neuroimmune Pharmacol 4, 419-429, doi: 10.1007/s11481-009-9176-0 (2009).
40 Su, X. et al. Synuclein activates microglia in a model of Parkinson's disease. Neurobiol Aging 29, 1690-1701, doi: 10.1016/j.neurobiolaging.2007.04.006 (2008).
41 Block, M. L. & Hong, J. S. Microglia and inflammation-mediated neurodegeneration: common mechanism. Prog Neurobiol 76, 77-98, multiple triggers with a doi: 10.1016/j.pneurobio.2005.06.004 (2005).
42 Kim, Y. S. & Joh, T. H. Microglia, major player in the brain inflammation: their roles in the pathogenesis of Parkinson's disease. Exp Mol Med 38, 333-347, doi: 10.1038/emm.2006.40 (2006).
43 Mackie, P. et al. The dopamine transporter: An unrecognized nexus for dysfunctional peripheral immunity and signaling in Parkinson's Disease. Brain Behav Immun 70, 21-35, doi: 10.1016/j.bbi.2018.03.020 (2018).
44 Braak, H. & Del Tredici, K. Invited Article: Nervous system pathology in sporadic Parkinson disease. Neurology 70, 1916-1925, doi: 10.1212/01.wnl.0000312279.49272.9f (2008).
45 Graham, F. L., Smiley, J., Russell, W. C. & Nairn, R. Characteristics of a human cell line transformed by DNA from human adenovirus type 5. J Gen Virol 36, 59-74, doi: 10.1099/0022-1317-36-1-59 (1977).
46 Goodwin, J. S. et al. Amphetamine and methamphetamine differentially affect dopamine transporters in vitro and in vivo. J Biol Chem 284, 2978-2989, doi: 10.1074/jbc.M805298200 (2009).
47 Saha, K. et al. Intracellular methamphetamine prevents the dopamine-induced enhancement of neuronal firing. Biol Chem 289, 22246-22257, doi: 10.1074/jbc.M114.563056 (2014).
48 Greene, L. A. & Tischler, A. S. Establishment of a noradrenergic clonal line of rat adrenal pheochromocytoma cells which respond to nerve growth factor. Proc Natl Acad Sci USA 73, 2424-2428, doi: 10.1073/pnas.73.7.2424 (1976).
49 Cartier, E. A. et al. A biochemical and functional protein complex involving dopamine synthesis and transport into synaptic vesicles. J Biol Chem 285, 1957-1966, doi: 10.1074/jbc.M109.054510 (2010).
50 Swant, J. et al. alpha-Synuclein stimulates a dopamine transporter-dependent chloride current and modulates the activity of the transporter. J Biol Chem 286, 43933-43943, doi: 10.1074/jbc.M111.241232 (2011).
51 Gopinath, A. et al. A novel approach to study markers of dopamine signaling in peripheral immune cells. J Immunol Methods 476, 112686, doi: 10.1016/j.jim.2019.112686 (2020).
52 Corkum, C. P. et al. Immune cell subsets and their gene expression profiles from human PBMC isolated by Vacutainer Cell Preparation Tube (CPT™) and standard density gradient. BMC Immunol 16, 48, doi: 10.1186/s12865-015-0113-0 (2015).
53 Flierl, M. A., Rittirsch, D., Huber-Lang, M., Sarma, J. V. & Ward, P. A. Catecholamines-crafty weapons in the inflammatory arsenal of immune/inflammatory cells or opening pandora's box? Mol Med 14, 195-204, doi: 10.2119/2007-00105.Flierl (2008).
54 Flierl, M. A. et al. Upregulation of phagocyte-derived catecholamines augments the acute inflammatory response. PLOS One 4, e4414, doi: 10.1371/journal.pone.0004414 (2009).
55 Torres, K. C. et al. Norepinephrine, dopamine and dexamethasone modulate discrete leukocyte subpopulations and cytokine profiles from human PBMC. J Neuroimmunol 166, 144-157, doi: 10.1016/j.jneuroim.2005.06.006 (2005).
56 Kustrimovic, N. et al. Dopaminergic Receptors on CD4+ T Naive and Memory Lymphocytes Correlate with Motor Impairment in Patients with Parkinson's Disease. Sci Rep 6, 33738, doi: 10.1038/srep33738 (2016).
57 Bergquist, J., Tarkowski, A., Ekman, R. & Ewing, A. Discovery of endogenous catecholamines in lymphocytes and evidence for catecholamine regulation of lymphocyte function via an autocrine loop. Proc Natl Acad Sci USA 91, 12912-12916, doi: 10.1073/pnas.91.26.12912 (1994).
58 Eugenin, E. A., Gaskill, P. J. & Berman, J. W. Tunneling nanotubes (TNT) are induced by HIV-infection of macrophages: a potential mechanism for intercellular HIV trafficking. Cell Immunol 254, 142-148, doi: 10.1016/j.cellimm.2008.08.005 (2009).
59 Nolan, R. & Gaskill, P. J. The role of catecholamines in HIV neuropathogenesis. Brain Res 1702, 54-73, doi: 10.1016/j.brainres.2018.04.030 (2019).
60 Nolan, R. A., Muir, R., Runner, K., Haddad, E. K. & Gaskill, P. J. Role of Macrophage Dopamine Receptors in Mediating Cytokine Production: Implications for Neuroinflammation in the Context of HIV-Associated Neurocognitive Disorders. J Neuroimmune Pharmacol 14, 134-156, doi: 10.1007/s11481-018-9825-2 (2019).
61 Merry, K. & Gowen, M. The transcriptional control of TGF-beta in human osteoblast-like cells is distinct from that of IL-1 beta. Cytokine 4, 171-179, doi: 10.1016/1043-4666 (92) 90052-s (1992).
62 Pickel, V. M., Joh, T. H., Field, P. M., Becker, C. G. & Reis, D. J. Cellular localization of tyrosine hydroxylase by immunohistochemistry. J Histochem Cytochem 23, 1-12, doi: 10.1177/23.1.234988 (1975).
63 Kastner, A., Hirsch, E. C., Herrero, M. T., Javoy-Agid, F. & Agid, Y. Immunocytochemical quantification of tyrosine hydroxylase at a cellular level in the mesencephalon of control subjects and patients with Parkinson's and Alzheimer's disease. J Neurochem 61, 1024-1034, doi: 10.1111/j.1471-4159.1993.tb03616.x (1993).
64 Yan, H. Q. et al. Delayed increase of tyrosine hydroxylase expression in rat nigrostriatal system after traumatic brain injury. Brain Res 1134, 171-179, doi: 10.1016/j.brainres.2006.11.087 (2007).
65 Witkovsky, P., Gabriel, R. & Krizaj, D. Anatomical and neurochemical characterization of dopaminergic interplexiform processes in mouse and rat retinas. J Comp Neurol 510, 158-174, doi: 10.1002/cne.21784 (2008).
66 Giguere, N. et al. Increased vulnerability of nigral dopamine neurons after expansion of their axonal arborization size through D2 dopamine receptor conditional knockout. PLOS Genet 15, e1008352, doi: 10.1371/journal.pgen.1008352 (2019).
67 Colon-Perez, L. M. et al. Functional connectivity, behavioral and dopaminergic alterations 24 hours following acute exposure to synthetic bath salt drug methylenedioxypyrovalerone. Neuropharmacology 137, 178-193, doi: 10.1016/j.neuropharm.2018.04.031 (2018).
68 Contini, M. & Raviola, E. GABAergic synapses made by a retinal dopaminergic neuron. Proc Natl Acad Sci USA 100, 1358-1363, doi: 10.1073/pnas.0337681100 (2003).
69 Feinstein, P., Bozza, T., Rodriguez, I., Vassalli, A. & Mombaerts, P. Axon guidance of mouse olfactory sensory neurons by odorant receptors and the beta2 adrenergic receptor. Cell 117, 833-846, doi: 10.1016/j.cell.2004.05.013 (2004).
70 Shaw, G., Morse, S., Ararat, M. & Graham, F. L. Preferential transformation of human neuronal cells by human adenoviruses and the origin of HEK 293 cells. Faseb j 16, 869-871, doi: 10.1096/fj.01-0995fje (2002).
71 Miller, D. R. et al. Methamphetamine regulation of activity and topology of ventral midbrain networks. PLOS One 14, e0222957, doi: 10.1371/journal.pone.0222957 (2019).
72 Trudeau, L. E. et al. The multilingual nature of dopamine neurons. Prog Brain Res 211, 141-164, doi: 10.1016/b978-0-444-63425-2.00006-4 (2014).
73 Morales, M. & Margolis, E. B. Ventral tegmental area: cellular heterogeneity, connectivity and behaviour. Nature Reviews Neuroscience 18, 73-85, doi: 10.1038/nrn.2016.165 (2017).
74 Caggiu, E. et al. Inflammation, Infectious Triggers, and Parkinson's Disease. Front Neurol 10, 122, doi: 10.3389/fneur.2019.00122 (2019).
75 Kozina, E. et al. Mutant LRRK2 mediates peripheral and central immune responses leading to neurodegeneration in vivo. Brain 141, 1753-1769, doi: 10.1093/brain/awy077 (2018).
76 Rentzos, M. et al. Circulating interleukin-15 and RANTES chemokine in Parkinson's disease. Acta Neurol Scand 116, 374-379, doi: 10.1111/j.1600-0404.2007.00894.x (2007).
77 Brodacki, B. et al. Serum interleukin (IL-2, IL-10, IL-6, IL-4), TNFalpha, and INFgamma 77 concentrations are elevated in patients with atypical and idiopathic parkinsonism. Neurosci Lett 441, 158-162, doi: 10.1016/j.neulet.2008.06.040 (2008).
78 Dufek, M. et al. Serum inflammatory biomarkers in Parkinson's disease. Parkinsonism Relat Disord 15, 318-320, doi: 10.1016/j.parkreldis.2008.05.014 (2009).
79 Deleidi, M. & Gasser, T. The role of inflammation in sporadic and familial Parkinson's disease. Cell Mol Life Sci 70, 4259-4273, doi: 10.1007/s00018-013-1352-y (2013).
80 Jenei-Lanzl, Z. et al. Anti-inflammatory effects of cell-based therapy with tyrosine hydroxylase-positive catecholaminergic cells in experimental arthritis. Ann Rheum Dis 74, 444-451, doi: 10.1136/annrheumdis-2013-203925 (2015).
81 Miller, L. E., Grifka, J., Scholmerich, J. & Straub, R. H. Norepinephrine from synovial tyrosine hydroxylase positive cells is a strong indicator of synovial inflammation in rheumatoid arthritis. J Rheumatol 29, 427-435 (2002).
82 Doll, D. N., Rellick, S. L., Barr, T. L., Ren, X. & Simpkins, J. W. Rapid mitochondrial dysfunction mediates TNF-alpha-induced neurotoxicity. J Neurochem 132, 443-451, doi: 10.1111/jnc.13008 (2015).
83 Giustarini, G. et al. Tissue influx of neutrophils and monocytes is delayed during development of trovafloxacin-induced tumor necrosis factor-dependent liver injury in mice. J Appl Toxicol 38, 753-765, doi: 10.1002/jat.3585 (2018).
84 Nakai, Y., Hamagaki, S., Takagi, R., Taniguchi, A. & Kurimoto, F. Plasma concentrations of tumor necrosis factor-alpha (TNF-alpha) and soluble TNF receptors in patients with anorexia nervosa. J Clin Endocrinol Metab 84, 1226-1228, doi: 10.1210/jcem.84.4.5589 (1999).
85 Turner, D. A. et al. Physiological levels of TNFalpha stimulation induce stochastic dynamics of NF-kappaB responses in single living cells. J Cell Sci 123, 2834-2843, doi: 10.1242/jcs.069641 (2010).
86 Damas, P. et al. Tumor necrosis factor and interleukin-1 serum levels during severe sepsis in humans. Crit Care Med 17, 975-978, doi: 10.1097/00003246-198910000-00001 (1989).
87 Kim, K. H. & Sederstrom, J. M. Assaying Cell Cycle Status Using Flow Cytometry. Curr Protoc Mol Biol 111, 28.26.21-28.26.11, doi: 10.1002/0471142727.mb2806s111 (2015).
88 Pereira, J. R. et al. IL-6 serum levels are elevated in Parkinson's disease patients with fatigue compared to patients without fatigue. J Neurol Sci 370, 153-156, doi: 10.1016/j.jns.2016.09.030 (2016).
89 Seppi, K. et al. Update on treatments for nonmotor symptoms of Parkinson's disease—an evidence-based medicine review. Mov Disord 34, 180-198, doi: 10.1002/mds.27602 (2019).
90 Lindqvist, D. et al. Non-motor symptoms in patients with Parkinson's disease-correlations inflammatory cytokines with in serum. PLOS One 7, e47387, doi: 10.1371/journal.pone.0047387 (2012).
91 Joers, V. et al. Microglia, inflammation and gut microbiota responses in a progressive monkey model of Parkinson's disease: A case series. Neurobiol Dis 144, 105027, doi: 10.1016/j.nbd.2020.105027 (2020).
92 O'Reilly, M. L. et al. Pharmacological Inhibition of Soluble Tumor Necrosis Factor-Alpha Two Weeks after High Thoracic Spinal Cord Injury Does Not Affect Sympathetic Hyperreflexia. J Neurotrauma, doi: 10.1089/neu.2020.7504 (2021).

What is claimed is:

1. A method for detecting a level of tyrosine hydroxylase (TH) in a biosample from a subject, wherein the biosample comprises a homogenate of peripheral monocytes from the subject, the method comprising conducting an ELISA on the biosample using a biotinylated anti-TH antibody under conditions to allow the biotinylated anti-TH antibody to bind to TH, and administering to the subject an amount of a TNFα inhibitor effective to decrease TH in peripheral monocytes of the subject.

2. The method of claim 1, wherein the TNFα inhibitor comprises a dominant negative mutant of TNFα soluble form.

3. The method of claim 2, wherein the dominant negative mutant of TNFα soluble form is XPRO 1595.

4. The method of claim 1, wherein the administering step is conducted if the TH level in the biosample is at or higher than a threshold level.

5. The method of claim 4, wherein the threshold level comprises a level that is at least 10% higher than a biosample of peripheral monocytes from a healthy subject.

6. The method of claims 4 and 5, wherein the threshold level is at least 15 pg TH, at least 20 pg TH, at least 25 pg TH, at least 30 pg TH, at least 35 pg TH, at least 40 pg TH, at least 45 pg TH, at least 50 pg TH, at least 55 pg/TH, at least 60 pg/TH at least 70 pg TH, at least 80 pg TH, at least 90 pg TH, at least 100 pg/TH at least 125 pg/TH or at least 150 pg TH per mg protein in the biosample.

7. The method of any of claims 1-6, wherein the amount of the TNFα inhibitor is an amount effect to reduce TH in peripheral monocytes by at least 10, at least 20, at least 30, at least 40, at least 50, at least 60, at least 70, at least 80 or at least 90 percent.

8. The method of any of claims 1-7, further comprising, following the administering step, detecting a level of TH in a second biosample from the subject.

9. A method for treating innate immune dysfunction in a subject comprising administering an amount of a TNFα inhibitor effective to decrease TH in peripheral monocytes of the subject if peripheral monocytes of the subject comprise a threshold level of TH.

10. The method of claim 9, wherein the threshold level comprises a level that is at least 10% higher than a biosample of peripheral monocytes from a healthy subject.

11. The method of claim 9 or 10, wherein the threshold level is at least 15 pg TH, at least 20 pg TH, at least 25 pg TH, at least 30 pg TH, at least 35 pg TH, at least 40 pg TH, at least 45 pg TH, at least 50 pg TH, at least 55 pg/TH, at least 60 pg/TH at least 70 pg TH, at least 80 pg TH, at least 90 pg TH, at least 100 pg/TH at least 125 pg/TH or at least 150 pg TH per mg protein in the biosample.

12. The method of any of claims 9-11, further comprising detecting a level of TH in a biosample of the subject, wherein the biosample comprises a homogenate of peripheral monocytes.

13. The method of claim 12, wherein detecting occurs before or after administering, or both.

14. The method of any of claim 12 or 13, wherein detecting a level of TH comprises subjecting the biosample to an ELISA that implements a biotinylated anti-TH antibody.

15. The method of any of claims 9-14, wherein innate immune dysfunction is associated with a higher risk of developing a neurodegenerative disease or autoimmune disorder.

16. The method of claim 15, wherein the neurodegenerative disease is Alzheimer's disease or Parkinson's disease.

17. The method of any of claims 9-16, wherein the amount of the TNFα inhibitor is an amount effect to reduce TH in peripheral monocytes by at least 10, at least 20, at least 30, at least 40, at least 50, at least 60, at least 70, at least 80 or at least 90 percent relative to a pre-administered level.

18. A method comprising:
obtaining a homogenate of peripheral monocytes from a subject;
subjecting the homogenate to a biotinylated anti-Tyrosine hydroxylase (TH) antibody to form a homogenate antibody combination;
subjecting the combination to a horseradish peroxidase conjugated with avidin (HRP-avidin), wherein contact between HRP-avidin and biotinylated anti-TH antibody is made under conditions to produce a detectable signal;
measuring the detectable signal, wherein a signal meeting a threshold level indicates innate immune dysfunction in the subject.

19. The method of claim 18, wherein the threshold level comprises a level that is at least 10% higher than that in a homogenate of peripheral monocytes from a healthy subject.

20. The method of claim 18, wherein the threshold level is at least 15 pg TH, at least 20 pg TH, at least 25 pg TH, at least 30 pg TH, at least 35 pg TH, at least 40 pg TH, at least 45 pg TH, at least 50 pg TH, at least 55 pg/TH, at least 60 pg/TH at least 70 pg TH, at least 80 pg TH, at least 90 pg TH, at least 100 pg/TH at least 125 pg/TH or at least 150 pg TH per mg protein in the homogenate.

21. The method of any of claims 1-17, wherein the TNFα inhibitor is in a composition with a pharmaceutically acceptable carrier, excipient, vehicle, or diluent.

22. A method of any of claims 1-17 and 21, further comprising detecting TH in a second biosample obtained from the subject following the administering step.

23. The method of claim 22, further comprising adjusting dose amount or frequency of the TNFα based on a level of TH detected in the second biosample.

24. The method of claim 18, wherein the conditions to produce a detectable signal comprises contact in the presence of an HRP substrate.

25. The method of claim 24, wherein the detectable signal comprises detecting absorbance at 450 nm.

26. The method of claim 24, wherein the detectable signal is a colorimetric signal.
